(19) United States
(12) Patent Application Publication
(10) Pub. No.: US 2020/0108115 A1
(43) Pub. Date: Apr. 9, 2020

(54) MEDICINAL COMPOSITION DERIVED FROM MULTIPLE PLANT SOURCES FOR GASTROINTESTINAL DISORDER

(71) Applicant: BENNY ANTONY, ANGAMALY (IN)

(72) Inventor: BENNY ANTONY, ANGAMALY (IN)

(21) Appl. No.: 16/497,031

(22) PCT Filed: Mar. 27, 2018
(86) PCT No.: PCT/IB2018/052080

§ 371 Date: Sep. 24, 2019

(30) Foreign Application Priority Data
Mar. 29, 2017 (IN) 201741011142

(51) Int. Cl. A61K 36/9068; A61K 36/75; A61K 36/67; A61K 36/484; A61K 36/9066; A61K 36/23; A61K 36/8905; A61K 36/736; A61K 36/8965; A61K 36/90; A61K 36/19; A61K 36/9062; A61K 31/192; A61K 31/704; A61P 1/00

(52) U.S. Cl. CPC A61K 36/9068; A61K 2236/333; A61K 36/67; A61K 36/484; A61K 36/9066; A61K 36/23; A61K 36/8905; A61K 36/736; A61K 36/8965; A61K 36/90; A61K 36/19; A61K 36/9062; A61K 31/192; A61K 31/704; A61P 1/00; A61K 2236/15; A61K 2236/13; A61K 36/75

(57) ABSTRACT

A gastrointestinal health enhancing composition having an extract of Piper longum, an extract of Terminalia chebula, an extract of Murraya koenigii, an extract Zingiber officinale and an extract Glycyrrhiza glabra. Gastrointestinal health enhancing compositions having additional extracts such as secondary and/or tertiary constituents. Methods of preparing the gastrointestinal health enhancing compositions. Methods of treating gastrointestinal disorders by administering the gastrointestinal health enhancing composition.

### FIELD OF THE INVENTION

The disclosure provides a medicinal composition useful in the treatment of gastrointestinal conditions. Said composition is derived from the extracts of various parts of different varieties of plants blended together. A method of preparation of the said medicinal composition also is disclosed here.

### BACKGROUND

The gastrointestinal system means the gastrointestinal tract plus accessory organs. The gastrointestinal tract consist of the oesophagus, stomach, small intestine, large intestine, rectum, and finally to the anus where undigested food is expelled. The accessory organs include the salivary glands, pancreas and liver. These secrete important enzymes into the digestive tract. The gall bladder, which stores bile, is also considered part of the gastrointestinal system.

Gastrointestinal conditions and disorders include but not limited to Gastroesophageal Reflux Disease (GERD), abdominal pain, indigestion, Gas in the gastrointestinal tract, fatigue, dehydration, gastrointestinal bleeding, and loss of appetite, flatulence and regurgitation. Symptoms related to gastrointestinal problems are acid reflex, heartburn, bloating, dyspepsia, vomiting, and irritable bowel movement. One or more symptoms or disorder may be observed in a single patient. They may be mild, self-limiting and temporary or may persist over a long term and affect health. Gastrointestinal problems or gut related disorder affect social and physiological health. Social and physiological health of a person determines the quality of life one live.

Functional gastrointestinal (GI) disorders affect millions of people of all ages; men, women, and children. They are the most commonly presented GI illnesses seen by doctors in primary care. The social and economic costs of gastrointestinal disorders are enormous. Not acknowledging Functional GI disorders and neglecting the symptoms of these disorders can cause discomfort, ranging from inconvenience to deep personal distress. For those with severe symptoms, the disorders can be debilitating, leaving them unable to fully participate in life and work.

The term “functional” is generally applied to disorders where the body's normal activities in terms of the movement of the intestines, the sensitivity of the nerves of the intestines, or the way in which the brain controls some of these functions is impaired. However, there are no structural abnormalities that can be seen by endoscopy, x-ray, or blood tests. Thus it is identified by the characteristics of the symptoms and infrequently, when needed, by carrying out limited tests. Functional disorders are those in which the bowel looks normal but doesn't function properly. They are the most common problems affecting the colon and rectum, and include constipation, Dyspepsia, Vomiting, Abdominal Pain, Belching and irritable bowel syndrome (IBS).

Irritable bowel syndrome (IBS) is characterized by chronic or recurrent abdominal pain and an erratic disturbance of defecation in the absence of detectable structural or biochemical abnormalities. Symptoms consistent with IBS are reported by about 15% of the general population, and IBS may account for at least 40% of referrals to gastroenterologists (Roger Jones, Susan Lydeard, Irritable bowel syndrome in the general population). Only some people with IBS in the community ever present for medical care. Symptom severity explains little of this behaviour. Traditionally, irritable bowel syndrome has been a diagnosis of exclusion, made only after organic disease has been ruled out by investigation.

Gastrointestinal Symptom Rating Scale: The GSRS, which has also previously been used, was originally constructed in analogy with the Comprehensive Psychological Rating Scale (CPRS). This is a rating scale for measuring changes in psychopathology. On the basis of clinical experience and reports in the literature on gastrointestinal symptoms of patients with irritable bowel syndrome and peptic ulcer disease, a selection of clinically relevant items were made.

Constipation is a common problem that affects up to 20% of the world's population. The prevalence of constipation is higher in women and in adults over the age of 65 years (Higgins and Johanson, 2004). It significantly affects quality of life, and leads to a loss of work productivity and abstinence from school (Dennison et al., 2005). Only about one-third of constipated patients seek medical care, and many patients self-treat their symptoms either by increasing fibre intake or by using over-the-counter laxatives (Rao et al., 2007).

The Rome Foundation was established in 1991 by Drossman et al, primarily to standardize consensus-derived criteria of functional gastrointestinal disorder, and released the Rome III criteria in 2006 for constipation.

Low fibre intake, inadequate hydration, reduced mobility as the result of general functional decline and institutionalization, reduced sensation of thirst, electrolyte disturbances (hypercalcemia, hypokalemia, hypermagnesemia), endocrine and metabolic disorders (e.g., diabetes mellitus, hyperparathyroidism, hypothyroidism, chronic renal failure), neurological disorders (e.g., dementia, Parkinson disease, neuropathies, multiple sclerosis, spinal cord injuries, cauda equine syndrome), psychological comorbidities (e.g., depression, distress, personality disorders, or history of abuse), and concurrent medications (e.g., anticholinergics, diuretics, β-blockers, opiates, iron supplements, calcium channel blockers, antidepressants, acetaminophen, aspirin and NSAIDs) all are said to contribute to chronic constipation, especially in the elderly (Bouras and Tangalos, 2009; Campbell et al., 1993; Towers et al., 1994; Nehra et al., 2000; Chang et al., 2007). Among them, the trio of insufficient dietary fibre, fluids, and exercise has been widely ascribed.

Normal defecation requires a series of orchestrated actions, starting with relaxation of the puborectalis muscles, descent of the pelvic floor with straightening of the anorectal angle, inhibition of segmental colonic peristalsis, contraction of the abdominal wall muscles, and finally, relaxation of the external anal sphincter with expulsion of faeces. Intrinsic factors leading to chronic constipation can be broadly classified into 2 categories: pelvic floor dysfunction (PFD) and slow colon transit time (STC). A retrospective study reported the prevalence of PFD as 37% and STC as 23% (Surrenti et al., 1995), based on physiologic tests. However, a clear distinction between the two is often impossible, with an overlap of up to 55% (Surrenti et al., 1995; Prather, 2004).

Anorectal/PFD involves laxity of the pelvic floor muscles, impaired rectal sensation, and decreased luminal pressure in the anal canal and has been documented as causes for chronic constipation in the elderly, especially in women (Bannister et al., 1987; Laurberg and Swash, 1989; Akervall et al., 1990). On the contrary, paradoxical contraction of the puborectalis and external anal sphincter during defecation can lead to incomplete emptying or even functional outlet obstruction. Finally, anatomic anomalies (such as rectal wall prolapse or rectocele) or perineal damage (from traumatic childbirth or sacral nerve injury) can also distort the normal functions of the anorectal/pelvic floor unit (Rao et al., 1998; Rao, 2009).

STC is a poorly understood condition thought to be a cause of intractable constipation in children (Hutson et al., 2001) and young women (Preston and Lennard-Jones, 1986). It is characterized by reduced high-amplitude propagated contractions (Herve et al., 2004; Bassotti et al., 1988) in the colon, leading to slow transit of faeces, bloating, abdominal discomfort, and infrequent defecation (Stivland et al., 1991). The protracted time in the colon also renders the faeces hard and small and fails to mount sufficient rectal pressure to trigger the defecation reflex (Bannister et al., 1987).

There are multiple reasons associated with gut related dysfunction at the same time there are multiple symptoms and problems associated with gut dysfunction. Administering micro or laxative formulation might provide a temporary relief but do not guaranty a sustainable and healthy gut function. Gastrointestinal function determines our interaction with others and also our state of comfort, so it is important for one to have a well-functioning gut to live a satisfied life or at least a functional lifestyle.

In CN10594624A a mixture of dried vegetables and yogurts is disclosed, the application suggests the mixture improves the nutritional structure and gastrointestinal health. The composition profoundly relied on the probiotic features of Yogurt to inhibit pathogenic bacteria that affect the gastrointestinal health. The dried fruits that got mentioned were banana and strawberry; they are used for the nutritional benefits only. The disclosed compositions do not just rely on the nutritional aspects of the plants but also their ability to improve gastro intestinal health.

Another blend with probiotic features were disclosed in WO 2017/027601 Al, a blend of probiotic composition and periodic composition is disclosed for maintaining or improving the gastrointestinal health in humans. In the above publication, the probiotic composition comprises the bacterium Bacillus coagulans and wherein the probiotic composition comprises a plant-based fiber, a resistant starch and a short-chain oligosaccharide. A solution for gastrointestinal problems without the use of plant fibers is looked upon in the present application.

There are several natural remedies for some sort of gastrointestinal problem but the results are not consistent and many a time raw materials such as some fruit or vegetable parts for such natural remedies are not readily available to general public.

In U.S. Pat. No. 7,651,705B2 Glycyrrhiza glabra, in U.S. Pat. No. 8,557,302B2 Andrographis paniculata and in WO 2014/167405A1 Centella asiatica are made part of a composition for treatment of ulcer. However, they do not discuss other gastrointestinal problems or the development of intestinal micro flora.

In U.S. Pat. No. 7,658,954B2 Tinospora cordifolia and Andrographis paniculata along with few other plant parts are blended to make a composition that can be used for reducing fever. The document also suggests using the composition for stomach ache that accompanies fever.

To evaluate the improvement in the gastrointestinal disorder treatment can be manifested by improvement in quality of life (QOL) of a patient. Healthy body and healthy mind constitutes a good life. There are multiple factors that can be used to determine the quality of life for a person, in US 2002/0156084A1 by use of N-Benzyl Piperazine and analogues as Quality of life enhancers. Quality of life enhancer is defined as the weight loss supplement, appetite suppressant and anti-depressant.

In the US 8,748,481B2 Quality of life was determined in patients suffering from irritable bowel syndrome (IBS) by patents answering an IBS QOL questionnaire for a time period and their scores being compiled and analysed. A similar clinical trial was conducted some disclosed compositions but unlike U.S. Pat. No. 8,748,481B2, the trial did not use any synthetic compound for the treatment of gastric disorder.

### Objects

A composition made of all plant parts that can treat slow colon transit time, pelvic floor dysfunction, Constipation, abdominal pain, indigestion, gas in the gastrointestinal tract, fatigue, dehydration, gastrointestinal bleeding, and loss of appetite, flatulence and regurgitation is not disclosed in any of the prior arts. There are multiple aspects that modulate the function of a gastrointestinal system and a composition that can regulate the entire system is required. Composition that can regulate gastrointestinal problems those are generally go unnoticed but has great affect in the quality of life we live. The principal object is to provide a medicinal composition derived from the extracts of various plants. Such plant composition or a blend is made up of Primary constituents, one or more Secondary constituents, one or more Tertiary constituents. Primary constituents comprise of Piper longum Linn, Terminalia chebula, Murraya koenigii, Zingiber officinale and Glycyrrhiza glabra.

Another objective is to provide composition comprising Primary constituents, one or more Secondary constituents, and one or more Tertiary constituents.

A further aspect is providing a unique ratio between Primary constituent, Secondary constituent and Tertiary constituent for effective treatment of gastrointestinal disorder.

A further aspect is providing a blend of different constituents of the selected medicinal composition with probiotics for medical use.

A further aspect is to develop a process to manufacture the compositions from the raw material using solvent extraction methods.

### SUMMARY

Disclosure relates to a medicinal composition derived from the extracts of multiple plants. The medicinal composition is made of a primary constituent that includes Piper longum Linn, Terminalia chebula Retz, Murraya koenigii, Zingiber officinale, and Glycyrrhiza glabra. Other embodiments include secondary constituents and/or tertiary constituents besides the primary constituents' composition.

The disclosure also provides a medicinal composition where Primary constituents' composition are blended with one or more plants selected from the group of Secondary Constituents; Curcuma longa, Centella asiatica, Cyperus rotundus, Alpinia galangal, Acorus calamus, Tinospora cordifolia, Hemidesmus indicus, Cinnamomum tamala, Terminallia bellerica, Momordica charantia, Piper nigrum, Asparagus racemosus, Andrographis paniculata, Pterocarpus marsupium, Swertia chirata Buch, and Boerhavia diffusa.

The disclosure also provides a medicinal composition with Primary Constituents, Secondary constituents and one or more constituents selected from Tertiary constituents of; Ajowa (Ptychotis ajowan), Coriander satuvum, Asparagus officinalis, Camellia sinensis, Ocimum sanctum, Sesamum indicum, Punica granatum, Emblica officinalis, Phyllanthus niruri, and Cassia fistula.

The ratio between Primary constituents to the rest of the composition is about 1:4 to 1:1.

The disclosure provides a dosage form having suitable excipients blended with other medicinal compositions. Medicinal composition is made in to a fixed dosage form. The dose can be used for gastrointestinal conditions.

Some aspects are directed towards a blend of medicinal composition with probiotic in a specific ratio. In some embodiment the probiotics used for the blend are for gastrointestinal conditions.

Some aspects are directed towards a medical composition capable of improving gastrointestinal condition and gastrointestinal problems.

Some aspects are directed towards a process to make medicinal composition by extracting the plant parts from Primary constituents. The process is also directed towards fixing the final polyphenol concentration in the composition to achieve a standardised product.

Some embodiments provide a gastrointestinal health enhancing composition having an extract of Piper longum, an extract of Terminalia chebula, an extract of Murraya koenigii, an extract Zingiber officinale and an extract Glycyrrhiza glabra. In some embodiments, the gastrointestinal health enhancing composition includes by weight: about 20% to about 30% of the extract of Piper longum, about 20% to about 30% of the extract of Terminalia chebula, about 20% to about 30% of extract of Zingiber officinale, about 20% to about 30% of extract of Glycyrrhiza glabra, and, about 1% to about 5% of extract of Murraya koenigii.

In some embodiments, the gastrointestinal health enhancing composition includes a weight ratio of 1:1:1:1 of the extract of Piper longum: the extract of Terminalia chebula: the extract of Zingiber officinale: the extract of Glycyrrhiza glabra. In some embodiments, the gastrointestinal health enhancing composition includes a weight ratio of the extract of Piper longum to extract of Murraya koenigii ranging from about 5:1 to about 25:1. In some embodiments, the gastrointestinal health enhancing composition includes a weight ratio of extract of Terminalia chebula to extract of Murraya koenigii ranging from about 5:1 to about 25:1. In some embodiments, the gastrointestinal health enhancing composition includes a weight ratio of extract of Zingiber officinale to extract of Murraya koenigii ranging from about 5:1 to about 25:1. In some embodiments, the gastrointestinal health enhancing composition includes a weight ratio of extract of Glycyrrhiza glabra to extract of Murraya koenigii ranging from about 5:1 to about 25:1. In some embodiments, the gastrointestinal health enhancing composition includes about 1% to about 10% alkaloids by weight. In some embodiments, the gastrointestinal health enhancing composition includes about 1% to about 10% gallic acid. In some embodiments, the gastrointestinal health enhancing composition includes about 10% to about 30% polyphenols. In some embodiments, gastrointestinal health enhancing composition includes about 0.1% to about 10% glycyrrhizic acid. In some embodiments, the gastrointestinal health enhancing composition includes about 0.1% to about 10% total gingerols. In some embodiments, the gastrointestinal health enhancing composition includes about 15% polyphenols. In some embodiments, the gastrointestinal health enhancing composition includes probiotics.

In some embodiments, the gastrointestinal health enhancing composition(primary constituent) further includes a secondary constituent which can be an extract of Curcuma longa, an extract of Centella asiatica, an extract of Cyperus rotundus, an extract of Alpinia galangal, an extract of Acorus calamus, an extract of Tinospora cordifolia, an extract of Hemidesmus indicus, an extract of Cinnamomum tamala, an extract of Terminallia bellerica, an extract of Momordica charantia, an extract of Piper nigrum, an extract of Asparagus racemosus, an extract of Andrographis paniculate, an extract of Pterocarpus marsupium, an extract of Swertia chirata Buch, an extract of Boerhavia diffusa, or combinations thereof.

In some embodiments, a gastrointestinal health enhancing composition having the primary constituents and the secondary constituents further includes tertiary constituents which can be an extract of Ajowa, an extract of Coriander satuvum, an extract of Asparagus officinalis, an extract of Camellia sinensis, an extract of Ocimum sanctum, an extract of Sesamum indicum, an extract of Punica granatum, an extract of Emblica officinalis, an extract of Phyllanthus niruri, an extract of Cassia fistula, or combinations thereof.

In some embodiments, in addition to the primary constituents a secondary constituent can be an extract of Curcuma longa, an extract of Centella asiatica, an extract of Cyperus rotundus, an extract of Terminallia bellerica, an extract of Hemidesmus indicus, an extract of Piper nigrum, an extract of Asparagus racemosus, an extract of Andrographis paniculata, an extract of Alpinia galangal, or combinations thereof.

In some embodiments, a gastrointestinal health enhancing composition includes by weight about 5% to about 15% of an extract of Piper longum, about 1% to about 11% of an extract of Terminalia chebula, about 1% to about 7% of an extract of Murraya koenigii, about 5% to about 15% of an extract of Zingiber officinale, about 1% to about 11% of an extract of Glycyrrhiza glabra, about 5% to about 15% of an extract of Curcuma longa, about 1% to about 11% of an extract of Centella asiatica, about 1% to about 11% of an extract of Cyperus rotundus, about 1% to about 11% of an extract of Terminallia bellerica, about 1% to about 11% of an extract of Hemidesmus indicus, about 1% to about 11% of an extract of Piper nigrum, about 1% to about 11% of an extract of Asparagus racemosus, about 1% to about 11% of an extract of Andrographis paniculata, and, about 1% to about 11% of an extract of Alpinia galangal.

In some embodiments, the gastrointestinal health enhancing composition further includes an extract of Curcuma longa, an extract of Centella asiatica, an extract of Cyperus rotundus, an extract of Tinospora cordifolia, an extract of Hemidesmus indicus, an extract of Andrographis paniculata, an extract of Alpinia galanga, an extract of Boerhavia diffusa, an extract of Cassia fistula, or combinations thereof.

In some embodiments, a gastrointestinal health enhancing composition includes by weight: about 1% to about 11% of an extract of Piper longum, about 1% to about 11% of an extract of Terminalia chebula, about 1% to about 11% of an extract of Murraya koenigii, about 1% to about 11% of an extract of Zingiber officinale, about 1% to about 11% of an extract of Glycyrrhiza glabra, about 1% to about 11% of an extract of Curcuma longa, about 1% to about 11% of an extract of Centella asiatica, about 1% to about 11% of an extract of Cyperus rotundus, about 1% to about 11% of an extract of Tinospora cordifolia, about 1% to about 11% of an extract of Hemidesmus indicus, about 1% to about 11% of an extract of Andrographis paniculata, about 1% to about 11% of an extract of Alpinia galanga, about 1% to about 11% of an extract of Boerhavia diffusa, and about 1% to about 11% of an extract of Cassia fistula.

In some embodiments, the extract of Piper longum is prepared from fruit of Piper longum. In some embodiments, the extract of Terminalia chebula is prepared from fruit of Terminalia chebula. In some embodiments, the extract of Murraya koenigii is prepared from leaf of Murraya koenigii. In some embodiments, the extract of Zingiber officinale is prepared from rhizomes of Zingiber officinale. In some embodiments, the extract of Glycyrrhiza glabra is prepared from rhizomes of Glycyrrhiza glabra.

Some embodiments provide a dosage form of the gastrointestinal health enhancing composition. The dosage form can be capsule, tablet, mini tablet, granule, sachet, powder, paste, ampoule, solution, suspension, emulsion, pills or cream.

Some embodiments provide product of the gastrointestinal health enhancing compositions. The product can be cookies, bread, or health supplement drinks.

Disclosure teaches methods of treating a gastrointestinal disorder by administering a disclosed gastrointestinal health enhancing composition to a subject in need thereof. The gastrointestinal disorder can be abdominal pain, gastric bloating (abdominal distension), nausea/vomiting, eructation, flatus, borborygmus, acid reflux, tightness in upper abdomen, heart burn or pain while passing stools or incomplete evacuation, inability to control the urgency of passing stools, difficulty in passage of stools, constipation, diarrhea syndrome, or indigestion syndrome.

Some embodiments teach methods of improving quality of life by administering a gastrointestinal health enhancing composition to a subject in need thereof. An improvement in the quality of life of the subject is assessed by improvement in a condition such as abdominal pain, gastric bloating, abdominal distension, nausea/vomiting, eructation, flatus, borborygmus, acid reflux, tightness in upper abdomen, heart Bum, pain while passing stools or incomplete evacuation, inability to control the urgency of passing stools, difficulty in passage of stools, constipation, diarrhea syndrome, or indigestion syndrome.

Some embodiments provide a method of improving quality of life by administering the gastrointestinal health enhancing composition. An improvement in the quality of life of the subject is assessed by questionnaire based on a scale such as gastrointestinal symptom rating scale, world health organization quality of life or patient assessment of constipation-quality of life.

Some embodiments provide a method of preparing a first gastrointestinal health enhancing composition. The method includes collecting fresh rhizomes of Zingiber officinale and roots of Glycyrrhiza glabra. Followed by washing the rhizomes and chopping into flakes. Next, drying the flakes to obtain dried flakes. Then extracting dried flakes with 90% methanol and collecting the solvent part of methanol extraction. Followed by concentrating the solvent part of the methanol extraction and drying the concentrated solvent part to obtain a powder of an alcoholic extract of Zingiber officinale and Glycyrrhiza glabra. The method further includes collecting fresh fruits of Piper longum and Terminalia chebula. Next, washing and chopping the fresh fruits into flakes. Followed by drying the flakes. Then extracting the flakes with 95% methanol and collecting the solvent part of the methanol extraction. Next, filtering and drying the concentrated solvent part of the methanol extraction to obtain a powder of an alcoholic extract of fruits of Piper longum and Terminalia chebula. The method further includes collecting fresh leaves of Murraya koenigii. Then washing and chopping the fresh leaves to obtain chopped leaves. Next drying the chopped leaves to obtain dried flakes. Followed by extracting the dried flakes with 95% methanol and collecting the solvent part of the methanol extraction. Next, filtering, concentrating and drying the concentrated solvent part of the methanol extraction to obtain a powder of an alcoholic extract of leaves of Murraya koenigii. The method further includes blending the powder of the alcoholic extract of Zingiber officinale and Glycyrrhiza glabra with the powder of powder of the alcoholic extract of fruits of Piper longum and Terminalia chebula and with the powder of the alcoholic extract of leaves of Murraya koenigii to obtain a first gastrointestinal health enhancing composition.

Some embodiments provide a method for preparing second gastrointestinal health enhancing composition. The method includes collecting fresh rhizomes of Centella asiatica, Curcuma longa, Cyperus rotundus, and Alpinia galangal, washing the rhizomes and chopping into flakes. Next, drying the flakes to obtain dried flakes. Followed by extracting dried flakes with 90% methanol and collecting the solvent part of methanol extraction. Then concentrating the solvent part of the methanol extraction and drying the concentrated solvent part to obtain a powder of an alcoholic extract of Centella asiatica, Curcuma longa, Cyperus rotundus, and Alpinia galangal; collecting fresh stem, bark and leaf of Tinospora cordifolia, Andrographis paniculata, Cassia fistula, Boerhaavia diffusa, and Centella asiatica. The method washing the fresh stem, bark and leaf and chopping into flakes. Next, drying the flakes to obtain dried flakes. Followed by extracting dried flakes with 90% methanol and collecting the solvent part of methanol extraction. Next, concentrating the solvent part of the methanol extraction and drying the concentrated solvent part to obtain a powder of an alcoholic extract of fresh stems, bark and leaves of Tinospora cordifolia, Andrographis paniculata, Cassia fistula, Boerhaavia diffusa, and Centella asiatica, mixing the powder of the alcoholic extract of Centella asiatica, Curcuma longa, Cyperus rotundus, and Alpinia galangal with the powder of the alcoholic extract of fresh stems, bark and leaves of Tinospora cordifolia, Andrographis paniculata, Cassia fistula, Boerhaavia diffusa, and Centella asiatica to obtain a mixture. Then blending the first gastrointestinal health enhancing composition with the mixture to obtain the second gastrointestinal health enhancing composition.

Some embodiments provide a method of preparing a gastrointestinal health enhancing composition. The method includes collecting a plant part such as leaf, stem, bark, rhizome, root, fruit, or combinations thereof. Then washing the plant part and chopping into flakes and drying the flakes to obtain dried flakes. Followed by extracting dried flakes with a solvent and collecting the solvent part after extraction, concentrating the solvent part and drying to obtain a powder of an extract of the plant part. The leaf can be Murraya koenigii, Boerhaavia diffusa, Centella asiatica, and/or Andrographis paniculate. The stem can be Tinospora cordifolia and/or Andrographis paniculata. A bark can be Cassia fistula. A rhizome such as Hemidesmus indicus, Cyperus rotundus, Alpinia galanga, Curcuma longa, and/or Zingiber officinale, the root can be Glycyrrhiza glabra. A fruit such as Piper longum and/or Terminalia chebula. The solvent can be hydroalcoholic 70% methanol or ethyl acetate. The powder of the extract can be of an extract of the leaf of Murraya Koenigii, an extract of the leaf of Boerhavia diffusa, an extract of the leaf of Centella asiatica, an extract of the leaf of Andrographis paniculata, an extract of the stem of Tinospora cordifolia, an extract of the stem of Andrographis paniculata, an extract of the bark of Cassia fistula, an extract of the rhizome of Hemidesmus indicus, an extract of the rhizome of Cyperus rotundus, an extract of the rhizome of Alpinia galanga, an extract of the rhizome of Curcuma longa, an extract of the rhizome of Zingiber officinale, an extract of the root of Glycyrrhiza glabra, an extract of fruit of Piper longum, and an extract of fruit of Terminalia chebula. A first extract is obtained by combining the extract of leaf of Murraya Koenigii, extract of the rhizome of Zingiber officinale, and an extract of the root of Glycyrrhiza glabra, an extract of fruit of Piper longum, and an extract of fruit of Terminalia chebula.

Some embodiments provide a method of preparing a second extract by combining an extract of the leaf of Murraya Koenigii, an extract of the leaf of Boerhaavia diffusa, an extract of the leaf of Centella asiatica, an extract of the leaf of Andrographis paniculata, an extract of the stem of Tinospora cordifolia, an extract of the stem of Andrographis paniculata, an extract of the bark of Cassia fistula, an extract of the rhizome of Hemidesmus indicus, an extract of the rhizome of Cyperus rotundus, an extract of the rhizome of Alpinia galanga, an extract of the rhizome of Curcuma longa, an extract of the rhizome of Zingiber officinale, an extract of the root of Glycyrrhiza glabra, an extract of fruit of Piper longum, and an extract of fruit of Terminalia chebula.

Some embodiments provide a method of preparing a third extract by combining the extract of the leaf of Murraya Koenigii, an extract of the leaf of Centella asiatica, an extract of the leaf of Andrographis paniculata, an extract of the stem of Andrographis paniculata, an extract of the bark of Cassia fistula, an extract of the rhizome of Cyperus rotundus, an extract of the rhizome of Curcuma longa, an extract of the rhizome of Zingiber officinale, an extract of the root of Glycyrrhiza glabra, an extract of fruit of Piper longum, an extract of fruit of Terminalia chebula, an extract of fruit of Terminalia bellerica, an extract of fruit of Momordica charantica, an extract of fruit of Piper nigrum, and an extract of root of Asparagus racemosus.

Some embodiments are directed towards a process to prepare extract from Secondary constituents and Tertiary constituents through solvent extraction. The extract from Secondary constituents and Tertiary constituents are blended with Primary constituents extract in a specific ratio.

### BRIEF DESCRIPTION OF THE DRAWINGS

Various features and advantages of the disclosed teachings should become apparent from the following Figures of the accompanying drawings, which demonstrate the process steps we have developed during various process trials carried according to certain preferred embodiments as well as based on the findings.

FIG. 1: Process and steps of preparing medicinal composition from Primary constituents, solvent extraction is used, different extracts are blended together to get a composition.

FIG. 2: Process and step of preparing a Medicinal composition (P07) using Primary constituents, solvent extraction is used, different extracts are blended together with primary constituents' composition to get a medicinal composition (P07).

FIG. 3: Zone of inhibition study data (mm) for fourteen plant extracts against seven pathogenic bacteria strains are provided.

FIG. 4: Bar graph showing predicted ZOI of each constituent of a primary constituents' composition, a calculated prediction of the total ZOI of primary constituents' composition and the actual ZOI observed with the primary constituents' composition against Escherichia Coli.

FIG. 4 (CONT.): Bar graph showing predicted ZOI of each constituent of a primary constituents' composition, a calculated prediction of the total ZOI of primary constituents' composition and the actual ZOI of the primary constituents' composition against Staphylococcus aureus.

FIG. 5: Bar graph showing predicted ZOI of each constituent of a primary constituent composition, a calculated prediction of the total ZOI of primary constituents' composition and the actual ZOI of the primary constituents' composition against Vibrio cholera.

FIG. 5 (CONT.): Bar graph showing predicted ZOI of each constituent of a primary constituents' composition, a calculated prediction of the total ZOI of primary constituents' composition and the actual ZOI of the primary constituent composition against Helicobacter pylori.

FIG. 6: Bar graph showing predicted ZOI of each constituent of a primary constituents' composition, a calculated prediction of the total ZOI of primary constituent composition and the actual ZOI of the primary constituent composition against Salmonella typhimurium.

FIG. 6 (CONT.): Bar graph showing predicted ZOI of each constituent of a primary constituents' composition, a calculated prediction of the total ZOI of primary constituent composition and the actual ZOI of the primary constituent composition against Salmonella Klebsiella oxytoca.

FIG. 7: Bar graph showing predicted ZOI of each constituent of a primary constituents' composition, a calculated prediction of the total ZOI of primary constituents' composition and the actual ZOI of the primary constituents' composition against Shigella Dysenteriae.

FIG. 8: Four bar graphs showing ZOI against Escherichia Coli, Staphylococcus aureus. The Bar graph shows the actual ZOI of P07, P03 and their respective calculated prediction of the ZOI, compared to show difference in efficacy.

FIG. 8 (CONT.): Four bar graphs showing ZOI against Salmonella typhimurium and Klebsiella oxytoca. The Bar graph shows the actual ZOI of P07, P03 and their respective calculated prediction of the ZOI, compared to show difference in efficacy.

FIG. 9: Three bar graphs showing a total predicted ZOI against Shigella Dysenteriae and Helicobacter pylori. The Bar graph shows the actual ZOI of P07, P03 and their respective calculated prediction of the ZOI, compared to show difference in efficacy. The first and third bars show the predicted ZOI values. The second and fourth bars show the actual ZOI values.

FIG. 9 (CONT.): Three bar graphs showing a total predicted ZOI against Vibrio cholera. The Bar graph shows the actual ZOI of P07, P03 and their respective calculated prediction of the ZOI, compared to show difference in efficacy. The first and third bars show the predicted ZOI values. The second and fourth bars show the actual ZOI values.

FIG. 10: Table provides the Minimum Inhibitory Concentration (MIC) value of 16 plant parts and Primary constituents' composition, P07, P03 and controls.

FIG. 11: Provides two sets of graphs. The first set of graphs provides MIC value when primary constituents' composition of Piper longum Linn, Terminalia chebula Retz, Murraya koenigii, Zingiber officinale, and Glycyrrhiza glabra is administered to each of seven pathogenic bacterial strains (Escherichia Coli, Staphylococcus aureus, Salmonella typhimurium, Klebsiella oxytoca, Vibrio cholera, Shigella Dysenteriae or Helicobacter pylori). The second set of graphs show the MIC value of primary constituents' composition against five beneficial bacteria strain (Lactobacillus acidophilus, Streptococcus thermophilus, Bacteroides fragilis, Clostridium butyricum and Faecalibacterium prausnitzii). The actual MIC value obtained as well as expected (or predicted) MIC value when the primary constituents' composition is administered against a bacterial strain is also indicated as two differently shaded bars. The first bar gives the expected (or predicted value) and the second bar provides the actual value.

FIG. 12: Provides two sets of graphs. The first set of graphs shows the MIC values of P07 composition against seven pathogenic bacterial strains (Escherichia Coli, Staphylococcus aureus, Salmonella typhimurium, Klebsiella oxytoca, Vibrio cholera, Shigella Dysenteriae and Helicobacter pylori). The second set of graphs shows the MIC value of P07 against five beneficial bacteria strain (Lactobacillus acidophilus, Streptococcus thermophilus, Bacteroidesfragilis, Clostridium butyricum and Facalibacterium prausnitzii). The actual MIC value obtained as well as expected (or predicted) MIC value when the P07 composition is administered against a bacterial strain is also indicated as two differently shaded bars. The first bar shows the expected (or predicted or calculated value) and the second bar provides the actual value.

FIG. 13: Provides two sets of graphs. The first set of graphs shows MIC values when P03 composition was administered against seven pathogenic bacterial strains (Escherichia Coli, Staphylococcus aureus, Salmonella typhimurium, Klebsiella oxytoca, Vibrio cholera, Shigella Dysenteriae and Helicobacter pylori). The second set of graphs show the MIC value of P03 against five beneficial bacteria strain (Lactobacillus acidophilus, Streptococcus thermophilus, Bacteroidesfragilis, Clostridium butyricum and Facalibacterium prausnitzii). The actual MIC value obtained as well as expected (or predicted) MIC value when the P03 composition is administered against a bacterial strain is also indicated as two differently shaded bars. The first bar shows the expected (or predicted or calculated value) and the second bar provides the actual value.

FIG. 14: Process and step of preparing a Medicinal composition (P07) having 14 plant extracts. Provides solvent extraction used for each plant. 14 extracts are blended together to obtain a medicinal composition (P07).

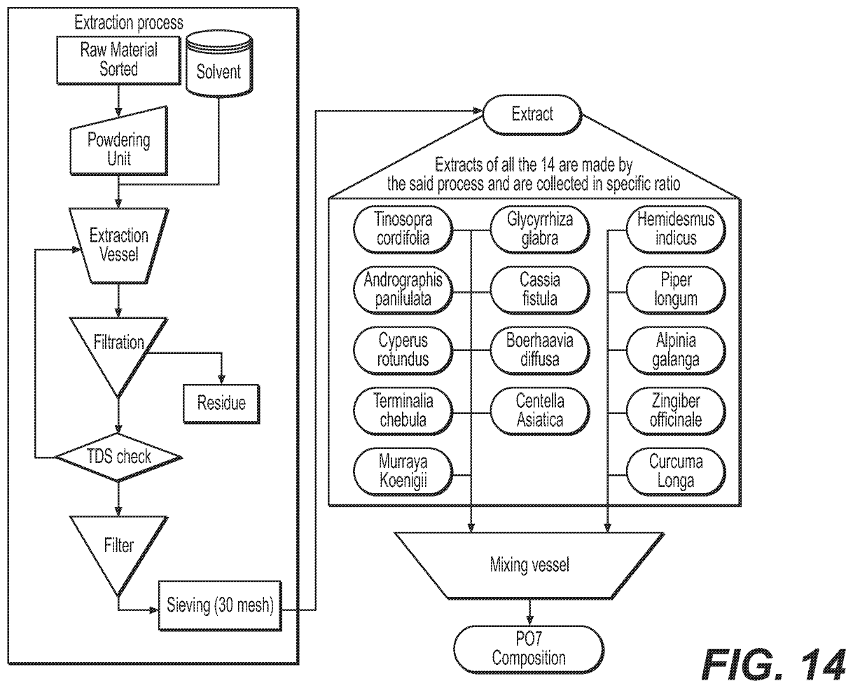

### DETAILED DESCRIPTION THE INVENTION

Disclosure provides medicinal compositions to improve the gastrointestinal health of a patient on administration as well as a method of preparation for said composition from extracts derived from various plants. The medicinal composition is in the form of a dietary supplement that will ensure a healthy gastric health when consumed regularly. The composition is a unique blend of extracts derived from various parts of plants that includes:

1. Piper longum (PL): Long pepper is a close relative of Piper nigrum, which gives black, green, and white pepper and has a similar but generally hotter flavour. The fruits contain the alkaloid piperine, which contributes to their pungency. Piperine is an alkaloid found naturally in plants belonging to the pyridine group of Piperaceae family. Piperine is the Trans stereoisomer of 1-piperoylpiperidine. It is richest in glutathione peroxidase and glucose-6-phosphate dehydrogenase, while vitamin E, vitamin A and exhibited strong antioxidant activity.

2. Terminalia chebula (TC): The plants of genus Terminalia, comprising of 250 species, are widely distributed in tropical parts of the world, fruits of Terminalia chebula Retzius (Combretaceae), commonly known as black Myroblans in English and Harad in Hindi. The three Terminalia trees (Terminalia chebula Retz, Terminalia bellerica and Terminalia horrida) belong to the family Combretaceae. Analysis of fruit showed hydrolyzable Gallic acid and simple gallate esters, Chebulic acid and chebulic ellagitannins, tannins, including simple gallate esters, ellagic acid derivatives and glycosides, and various ellagitannins.

3. Murraya koenigii (MK): The plant Murraya koenigii Spreng. Belonging to the family Rutaceae is native to India and now distributed in most of southern Asia. The leaves of this plant are well-known as curry leaves and have been used as one of the important ingredient of Indian cooking. The major constituents responsible for the aroma and flavor have been reported as pinene, sabinene, caryophyllene, cadinol and cadinene, β-pinene, β-caryophyllene and α-pinene, α-pinene, β-pinene and β-phellandrene, β-caryophyllene, aromadendrene and α-selinene, β-phellandrene, β-caryophyllene, α-pinene, (E)-β-ocimene and aromadendrene.

4. Ginger: Zingiber (ZO) (Zingiber officinale). The nutritional content of ginger includes protein, lipids, carbohydrates, minerals and vitamins plus trace nutrients. Ginger also has capsaicin, curcumin and limonene as well as proteolytic enzymes.

5. Glycyrrhiza glabra (GG): Licorice (Glycyrrhiza glabra L; Family: Papilionaceae/Fabaceae) is a traditional medicinal herb grows in the various parts of the world. It is a very sweet, moist, soothing herb that contains Flavonoids, Alkaloids, Steroids, Terpenoids, Saponins, Tannins, and Glycosides.

6. Curcuma longa (CL): Zingibera ceae is a perennial herb widely cultivated in tropical regions of Asia. Its rhizome is extensively used for imparting color and flavor to food. It is rich in curcumin, demethoxycurcumin, and bisdemethoxycurcumin Ar-turmerone, α-turmerone, and β-turmerone.

7. Centella asiatica (CA): It is a very important medicinal herb used in the orient, which is also becoming popular in the West. The herb is also used by the people of Java and other Indonesian islands. Analyses of the essential oil revealed monoterpenoid hydrocarbons, nine oxygenated monoterpenoids, sesquiterpenoid hydrocarbons, five oxygenated sesquiterpenoids, and one sulphide sesquiterpenoid.

8. Cyperus rotundus (CR): A cosmopolitan weed is found in all tropical, subtropical and temperate regions of the world. Nagarmotha (Cyperus rotundus) commonly known as Nagarmotha is found throughout India, it belongs to the family Cyperacea. The genus name Cyperus is derived from Cypeiros, which was the ancient Greek name for the genus, and rotundus is Latin word for round and refers to the tuber. The major chemical components of this herb are essential oils, flavonoids, terpenoids, sesquiterpenes, cyprotene, cyperene, aselinene, rotundene, valencene, cyperol, gurjunene, trans-calamenene, cadalene, cyperotundone, mustakone, isocyperol, acyperone, etc.

9. Tinospora cordifolia (TC): Menispermaceae, commonly known, as “Amrita” or “Guduchi” is an important drug of Indian Systems of Medicine ISM and used in medicines since times immemorial. Analysis shows Proteins, Carbohydrates, Phenols/Tannins, Flavonoids, Saponins, Steroids, and Terpenoids.

10. Hemidesmus indicus (HI): An Indian sarsaparilla (Anantamul) belonging to the family Asclepiadaceae, a twining shrub commonly found in India. The roots of the plant are woody and have a sweet taste, with cooling effect, and used in various ailment of diseases, a well-known drug in the Indian system of medicine.

11. Cinnamomum tamala (CT): It belonging to family Lauraceae is also known as Indian Cassia and the leaves are commonly called as bay leaves. It is found in South-east Asia, some Pacific Islands and Australia, growing mainly in tropical rain forests at varying altitudes.

12. Terminalia belerica (TB): It grows throughout the Indian subcontinent, Sri Lanka and the SE Asia. In the Indian system of medicine it has been known to be effective for gastroptosis.

13. Momordica charantia (MC): It is a climber belonging to family Cucurbitaceae, is commonly known as bitter gourd or bitter melon in English and karela in Hindi. Momordica means, “To bite” referring to the jagged edges of the leaf, which appear as if bitten. All parts of the plant, including the fruit, taste bitter. The fruit is oblong and resembles a small cucumber.

14. Piper nigrum (PN) (black pepper): Finds extensive use in Indian system of medicine and also a popular spice. A number of piperidine and pyrrolidine alkamides are known to occur in Piper nigrum, the most important being piperine.

15. Asparagus racemosus (AR): The genus Asparagus has been recently moved from the subfamily Asparagae in the family Liliaceae to a newly created family Asparagaceae. In the traditional medicine it is known to be used for natural balancing of acidic digestion

16. Andrographis paniculata (AP): The herb contains diterpenoids, flavonoids and polyphenols as the major bioactive components, further analysis reviled 14-deoxyandrographolide, 14-deoxy-11, 12-didehydroandrographolide, 14-deoxy-14, 15-didehydroandrographolide, 14-acetylandrographolide, and 19-O-acetylanhydroandrographolide.

17. Pterocarpus marsupium (PM): Papilionaceae is a deciduous tree commonly distributed in forests of the western ghats of India. The plant is commonly known as Asanahmbijakah, Red Kino tree or Honne. Pterocarpans are isoflavonoids which have a condensed dihydrofuran system between the heterocyclic and side phenyl rings, two pterocarpans (−)-homopterocarpin (I) and (−)-pterocarpin (II) were found to occur very commonly in Pterocarpus woods. Isoflavones from pterocarpus includes; Prunetin, Muningin, Formononetin, Santa, 7-O-Methylteetorigenin, 3′-Hydroxyformononetin and Pseudobaptigenin. Pterostilbene, Other constituents detected are liquiritigenin, isoliquiritigenin, Pterofuran, triterpene acid, acetyloleanolic acid, and volatile terpenes.

Other than the 17 plants described above the medicinal composition may also include constituents selected from Swertia chirata Buch, Boerhavia diffusa, Ajowa (Ptychotis ajowan), Coriander satuvum, Asparagus officianlis, Camellia sinensis, Ocimum sanctum, Sesamum indicum, Punica granatum, Emblica officinale, Phyllanthus niruri, Cassia fistula and Tinospora cordifolia.

Composition made from one or more constituents listed above or in table 1 will be referred to as “medicinal composition” throughout the specification, unless said otherwise and this medicinal composition is derived from the extracts of various parts of the plants. Plant will include weeds, shrubs, trees, bushes, grasses, vines, ferns, mosses, and green algae.

In some embodiment the medicinal composition can be water soluble, in some embodiment it is in water dispersible form, in yet another embodiment it can be lipid soluble, while in another embodiment medicinal composition is in liquid state, in some it is in powdered form.

Throughout the specification the dosage is for a person with an average body weight of 70 Kgs, the dosage may vary according to body weight of an individual.

Throughout the specification any percentage or ratio provided for the composition is on weight bases, unless said otherwise.

The constituents of medicinal composition are divided into Primary, Secondary and Tertiary constituents. The medicinal composition is Primary Constituents alone or Primary Constituents blended with one or more constituent from Secondary and Tertiary. A preferred composition of medicinal composition will contain over 10 to 14 constituents. A medicinal composition will have all the primary constituents and one or more constituents selected from Secondary and Tertiary constituents.

The medicinal composition can be administered orally in any of the desired dose forms, such as powder, dissolved in a solvent, capsule, tablet, or made part of any other dietary product such as cookies, bread, health supplement drinks.

Excipient referred in the discloser refers to anticaking agents, fillers, solvents, diluents, carrier, adsorbent and food products. The Table below list all constituents of medicinal composition in separate groups.

TABLE 1

| | PRIMARY | SECONDARY | TERTIARY |
|---|---|---|---|
| | CONSTITUENTS | CONSTITUENTS | CONSTITUENTS |
| 1 | Piper Longum | Curcuma Longa | Ajowa (Ptychotis |
| | | | ajowan) |
| 2 | Terminalia Chebula | Centella asiatica | Coriander satuvum |
| | Retz | | |
| 3 | Murraya koenigii | Cyperus rotundus | Asparagus |
| | | | officinalis |
| 4 | Zingiber officinale | Tinospora cordifolia | Camellia sinensis |
| 5 | Glycyrrhiza Glabra | Hemidesmus indicus | Ocimum sanctum |
| 6 | | Cinnamomum tamala | Sesamum indicum |
| 7 | | Terminallia bellerica | Punica granatum |
| 8 | | Momordica charantia | Emblica officinale |
| 9 | | Piper nigrum | Phyllanthus niruri |
| 10 | | Asparagus racemosus | Cassia fistula |
| 11 | | Andrographis | |
| | | paniculata | |
| 12 | | Pterocarpus | |
| | | marsupium | |
| 13 | | Alpinia galanga | |
| 14 | | Acorus calamus | |
| 15 | | Swertia chirata Buch | |
| 16 | | Boerhavia diffusa | |

One embodiment is a medicinal composition made up of Primary constituents, of which Piper longum extract makes up 20% to 30%, Terminalia chebula extract makes 20% to 30%, Zingiber officinale extract makes 20% to 30%, Glycyrrhiza glabra extract makes 20% to 30% and Murraya koenigii extract makes 1% to 5%. More preferably Piper longum, Terminalia chebula, Zingiber officinale and Glycyrrhiza glabra are in equal proportion, that is in 1:1:1:1 ratio. Each constituent is in a 5:1-25:1 ratio with Murraya koenigii, that is, any one of Piper longum Linn, Terminalia chebula, Zingiber officinale or Glycyrrhiza glabra is in 5:1-25:1 ratio with Murraya koenigii. More precisely extracts derived from fruit of Piper longum and Terminalia chebula, and leaf of Murraya koenigii and rhizomes of Zingiber officinale and Glycyrrhiza glabra. Piper longum Linn, Terminalia chebula, Murraya koenigii, Zingiber officinale and Glycyrrhiza glabra are the Primary constituents of the medicinal composition. The extracts are derived through solvent extraction and for extraction one or more solvents are selected from water, ethyl acetate, methanol, ethanol, or a combination there of.

Another aspect of the medicinal composition also includes any one or more constituents selected from Secondary constituent along with the Primary constituents. The Primary constituents make 25% to 95% of such medicinal composition.

In yet another embodiment Primary constituents along with one or more Secondary constituents are combined with one or more constituents from a group of Tertiary constituents. The Primary constituents make 25% to 50% of such medicinal composition.

Disclosure provides a medicinal composition which is a combination of Primary constituents with one or more constituents from Secondary and Tertiary constituents. Another aspect is the ratio between Primary constituents to rest of the plant constituents, which is about 1:4 to 1:1.

Disclosure teaches medicinal compositions which are a combination of Primary constituents, one or more constituents from Secondary constituents and one or more constituents from Tertiary constituents blended in a ratio of 1:1:1 to 5:9:3, preferably about 5:7:2.

Yet another aspect is a medicinal composition consisting of, Primary constituents 25% to 50% of composition, Secondary constituents about 28% to 64% and Tertiary constituents about 7% to 35%.

Yet another aspect is a dose form of medicinal composition. The dose form may be blended with suitable excipient. Medicinal compositions made in to dose form of 250 mg to 2000 mg, preferably in 500 mg to 1000 mg. The dose form can be selected from a group of but not limiting to capsule, tablet, mini tablet, granule, sachet, powder, paste, ampoule, solution, suspension, emulsion, pills or cream.

Some embodiments provide a gastrointestinal health enhancing composition having an extract of Piper longum, an extract of Terminalia chebula, an extract of Murraya koenigii, an extract Zingiber officinale and an extract Glycyrrhiza glabra. In some embodiments, the gastrointestinal health enhancing composition includes by weight: about 20% to about 30% of the extract of Piper longum, about 20% to about 30% of the extract of Terminalia chebula, about 20% to about 30% of extract of Zingiber officinale, about 20% to about 30% of extract of Glycyrrhiza glabra, and, about 1% to about 5% of extract of Murraya koenigii.

In some embodiments, the gastrointestinal health enhancing composition includes a weight ratio of 1:1:1:1 of the extract of Piper longum: the extract of Terminalia chebula: the extract of Zingiber officinale: the extract of Glycyrrhiza glabra. In some embodiments, the gastrointestinal health enhancing composition includes a weight ratio of the extract of Piper longum to extract of Murraya koenigii ranging from about 5:1 to about 25:1. In some embodiments, the gastrointestinal health enhancing composition includes a weight ratio of extract of Terminalia chebula to extract of Murraya koenigii ranging from about 5:1 to about 25:1. In some embodiments, the gastrointestinal health enhancing composition includes a weight ratio of extract of Zingiber officinale to extract of Murraya koenigii ranging from about 5:1 to about 25:1. In some embodiments, the gastrointestinal health enhancing composition includes a weight ratio of extract of Glycyrrhiza glabra to extract of Murraya koenigii ranging from about 5:1 to about 25:1.

In some embodiments, the gastrointestinal health enhancing composition includes about 1% to about 10% alkaloids by weight. In some embodiments, the gastrointestinal health enhancing composition includes about 1% to about 10% gallic acid. In some embodiments, the gastrointestinal health enhancing composition includes about 10% to about 30% polyphenols. In some embodiments, gastrointestinal health enhancing composition includes about 0.1% to about 10% glycyrrhizin acid. In some embodiments, the gastrointestinal health enhancing composition includes about 0.1% to about 10% total gingerols. In some embodiments, the gastrointestinal health enhancing composition includes about 15% polyphenols. In some embodiments, the gastrointestinal health enhancing composition includes probiotics.

In some embodiments, the gastrointestinal health enhancing composition (primary constituent) further includes a secondary constituent which can be an extract of Curcuma longa, an extract of Centella asiatica, an extract of Cyperus rotundus, an extract of Alpinia galangal, an extract of Acorus calamus, an extract of Tinospora cordifolia, an extract of Hemidesmus indicus, an extract of Cinnamomum tamala, an extract of Terminallia bellerica, an extract of Momordica charantia, an extract of Piper nigrum, an extract of Asparagus racemosus, an extract of Andrographis paniculate, an extract of Pterocarpus marsupium, an extract of Swertia chirata Buch, an extract of Boerhavia diffusa, or combinations thereof.

In some embodiments, a gastrointestinal health enhancing composition having the primary constituents and the secondary constituent further includes tertiary constituent which can be an extract of Ajowa, an extract of Coriander satuvum, an extract of Asparagus officinalis, an extract of Camellia sinensis, an extract of Ocimum sanctum, an extract of Sesamum indicum, an extract of Punica granatum, an extract of Emblica officinalis, an extract of Phyllanthus niruri, an extract of Cassia fistula, or combinations thereof.

In some embodiments, in addition to the primary constituents' composition a secondary constituent can be an extract of Curcuma longa, an extract of Centella asiatica, an extract of Cyperus rotundus, an extract of Terminallia bellerica, an extract of Hemidesmus indicus, an extract of Piper nigrum, an extract of Asparagus racemosus, an extract of Andrographis paniculata, an extract of Alpinia galangal, or combinations thereof.

In some embodiments, a gastrointestinal health enhancing composition includes by weight about 5% to about 15% of an extract of Piper longum, about 1% to about 11% of an extract of Terminalia chebula, about 1% to about 7% of an extract of Murraya koenigii, about 5% to about 15% of an extract of Zingiber officinale, about 1% to about 11% of an extract of Glycyrrhiza glabra, about 5% to about 15% of an extract of Curcuma longa, about 1% to about 11% of an extract of Centella asiatica, about 1% to about 11% of an extract of Cyperus rotundus, about 1% to about 11% of an extract of Terminallia bellerica, about 1% to about 11% of an extract of Hemidesmus indicus, about 1% to about 11% of an extract of Piper nigrum, about 1% to about 11% of an extract of Asparagus racemosus, about 1% to about 11% of an extract of Andrographis paniculata, and, about 1% to about 11% of an extract of Alpinia galangal.

In some embodiments, the gastrointestinal health enhancing composition further includes an extract of Curcuma longa, an extract of Centella asiatica, an extract of Cyperus rotundus, an extract of Tinospora cordifolia, an extract of Hemidesmus indicus, an extract of Andrographis paniculata, an extract of Alpinia galanga, an extract of Boerhavia diffusa, an extract of Cassia fistula, or combinations thereof.

In some embodiments, a gastrointestinal health enhancing composition includes by weight: about 1% to about 11% of an extract of Piper longum, about 1% to about 11% of an extract of Terminalia chebula, about 1% to about 11% of an extract of Murraya koenigii, about 1% to about 11% of an extract of Zingiber officinale, about 1% to about 11% of an extract of Glycyrrhiza glabra, about 1% to about 11% of an extract of Curcuma longa, about 1% to about 11% of an extract of Centella asiatica, about 1% to about 11% of an extract of Cyperus rotundus, about 1% to about 11% of an extract of Tinospora cordifolia, about 1% to about 11% of an extract of Hemidesmus indicus, about 1% to about 11% of an extract of Andrographis paniculata, about 1% to about 11% of an extract of Alpinia galanga, about 1% to about 11% of an extract of Boerhavia diffusa, and about 1% to about 11% of an extract of Cassia fistula.

In some embodiments, the extract of Piper longum is prepared from fruit of Piper longum. In some embodiments, the extract of Terminalia chebula is prepared from fruit of Terminalia chebula. In some embodiments, the extract of Murraya koenigii is prepared from leaf of Murraya koenigii. In some embodiments, the extract of Zingiber officinale is prepared from rhizomes of Zingiber officinale. In some embodiments, the extract of Glycyrrhiza glabra is prepared from rhizomes of Glycyrrhiza glabra.

Some embodiments provide a dosage form of the gastrointestinal health enhancing composition. The dosage form can be capsule, tablet, mini tablet, granule, sachet, powder, paste, ampoule, solution, suspension, emulsion, pills or cream.

Some embodiments provide product of the gastrointestinal health enhancing compositions. The product can be cookies, bread, or health supplement drinks.

Disclosure teaches methods of treating a gastrointestinal disorder by administering a disclosed gastrointestinal health enhancing composition to a subject in need thereof. The gastrointestinal disorder can be abdominal pain, gastric bloating (abdominal distension), nausea/vomiting, eructation, flatus, borborygmus, acid reflux, tightness in upper abdomen, heart bum, pain while passing stools or incomplete evacuation, inability to control the urgency of passing stools, difficulty in passage of stools, constipation, diarrhea syndrome, or indigestion syndrome.

Some embodiments teach methods of improving quality of life by administering a gastrointestinal health enhancing composition to a subject in need thereof. An improvement in the quality of life of the subject is assessed by improvement in a condition such as abdominal pain, gastric bloating, abdominal distension, nausea/vomiting, eructation, flatus, borborygmus, acid reflux, tightness in upper abdomen, heart Bum, pain while passing stools or incomplete evacuation, inability to control the urgency of passing stools, difficulty in passage of stools, constipation, diarrhea syndrome, or indigestion syndrome.

Some embodiments provide a method of improving quality of life by administering the gastrointestinal health enhancing composition. An improvement in the quality of life of the subject is assessed by questionnaire based on a scale such as gastrointestinal symptom rating scale, world health organization quality of life or patient assessment of constipation-quality of life.

A medicinal gastrointestinal health enhancing composition for the treatment of Gastrointestinal conditions is made of the plant composition contain Primary constituents, one or more plants from Secondary and Tertiary constituents.

Probiotics, which are live microbial ingredients, produce lactic and acetic acids and influence the peristalsis of intestines by reducing colonic pH, they play a significant role in regulating gut health. In one embodiment the medicinal composition is provided with probiotic bacteria, a composition with medicinal composition and probiotic bacteria. In another embodiment the probiotics are selected from a genus of Bifido bacterium, Lactobacillus, Streptococcus, Bacillus Coagulans, or combination. Medicinal composition is blended with probiotic in ratio 12:1 to 5:3. The medicinal composition with probiotic is provided in a dose form of 250 to 2000 mg, preferably in 500 to 1000 mg, more preferably 50 mg probiotics blended with about 400 mg medicinal composition.

Blend of medicinal composition with probiotic lowers abdominal pain, can decrease gastric bloating (abdominal distension), and reduce nausea/vomiting, eructation, flatus, borborygmus and acid reflux. Use of medicinal composition with probiotic decreases and manages gastrointestinal health by reducing Tightness in upper abdomen, Heart Burn, pain while passing stools or feeling of incomplete evacuation. Use of medicinal composition with probiotic reduces inability to control the urgency of passing stools, regulating passage of stool, decrease in hard stools, increased passage of stools, and loose stools.

In-vitro study conducted to compare the effect of Primary constituents' composition on bacteria and cell growth had shown that the whole of medicinal composition is superior in inhibiting pathogenic bacteria in comparison to its individual constituents. The effect the composition had on test samples during and after test is significantly better than one would expect from merely adding of each component. It is the synergy among the medicinal composition constituents that enable it to orchestrate a smooth gastrointestinal function.

Administering the medicinal composition regularly improves the health of stomach lining. Regular administration of medicinal composition for about a week shows improvement in the health of stomach lining. Administering the medicinal composition improves the health of mucous gland. The medicinal composition also expedites the recovery of damaged stomach lining. The medicinal composition can be used to treat peptic ulcer and intestinal infection or used along with drugs for treatment of peptic ulcer, or intestinal infection.

Another embodiment is a method to improve the gastrointestinal health of a patient by administering a medicinal composition derived from the Primary constituents. Medicinal composition lowers abdominal pain, decrease gastric bloating (abdominal distension); reduce nausea/vomiting, eructation, flatus, borborygmus and acid reflux. Medicinal composition decreases and manages gastrointestinal health by reducing Tightness in upper abdomen, Heart Burn, pain while passing stools or feeling of incomplete evacuation of stool. Use of medicinal composition reduces inability to control the urgency of passing stools, and regulating passage of stool. One embodiment medicinal compositions a combination of Alkaloid, Tannin, Saponin, Flavonoids, Terpenoids, Diterpenoids, and Triterpenoids derived from Primary constituents, Secondary Constituents and Tertiary constituents.

One aspect is a medicinal composition to improve gastrointestinal condition by administering Primary constituents, a composition with extract of Piper longum about 5% to 30%, Terminalia chebula 5% to 30%, Murraya koenigii 0.5% to 10%, Zingiber officinale 5% to 30% and Glycyrrhiza glabra 5% to 30%.

The medicinal composition is made up of active constituents such as Alkaloid, Polyphenol, Tannin, Terpenoids, Saponin, Flavanoids, and Triterpenoids. The medicinal composition is standardized with Polyphenol fixed at 15% of the composition. The process to make medicinal composition is also directed towards fixing the final polyphenol concentration in the composition to achieve a standardised product.

The medicinal composition is also made up of Glycyrrhizic acid, Piperine, Gallic acid, Curcuminoids, Andrographolide, and Gingerols Bitters. Glycyrrhizic acid makes up about 7% to 18%, Piperine makes up about 7% to 18%, Gallic acid makes up about 7% to 18%, Curcuminoids makes up about 7% to 18%, Andrographolide makes up about 7% to 18%, and Gingerols Bitters makes up about 7% to 18% of the composition.

In one embodiment, a medicinal composition, P01, is made up of about 7% to 20% Tinospora cordifolia, about 7% to 20% Hemidesmus indicus, about 7% to 20% Piper longum Linn, about 7% to 20% Alpinia galanga, about 7% to 20% Terminalia chebula Retz, about 7% to 20% Swertia chirata Buch, about 0.5% to 10% Murraya koenigii, about 7% to 20% Curcuma longa, and about 7% to 20% Zingiber officinale. Another aspect is that the product is blended with an excipient. The excipient can range from 15% to 60% of the total composition.

In yet another embodiment, a medicinal composition, P02, is made up of about 5% to 20% Piper longum Linn, 1% to 20% Terminalia chebula, 0.5% to 7% Murraya koenigii, 5% to 20% Zingiber officinale, 1% to 20% Glycyrrhiza glabra, 1% to 20% Centella asiatica, 1% to 20% Cyperus rotundus, 1% to 20% Tinospora cordifolia, 1% to 20% Hemidesmus indicus, 1% to 20% Cinnamomum tamala, 1% to 20% Alpinia galanga, 1% to 20% Acorns calamus, 1% to 20% Swertia chirata Buch, and 1% to 20% Boerhavia diffusa.

In yet another embodiment, a medicinal composition, P03, is made up of about 5% to 15% Piper longum Linn, about 1% to 11% Terminalia chebula Retz, about 1% to 7% Murraya koenigii, about 5% to 15% Zingiber officinale, about 1% to 11% Glycyrrhiza glabra, about 5% to 15% Curcuma longa, about 1% to 11% Centella asiatica, about 1% to 11% Cyperus rotundus, about 1% to 11% Terminallia bellerica about 1% to 11% Hemidesmus indicus, about 1% to 11% Piper nigrum, about 1% to 11% Asparagus racemosus, about 1% to 11% Andrographis paniculata, about 1% to 11% Alpinia galanga.

In yet another embodiment, a medicinal composition, P04, is made up of about 1% to 11% Piper longum Linn, about 1% to 11% Terminalia chebula Retz, about 1% to 11% Murraya koenigii, about 1% to 11% Zingiber officinale, about 1% to 11% Glycyrrhiza glabra, about 1% to 11% Curcuma longa, about 1% to 11% Centella asiatica, about 1% to 11% Cyperus rotundus, about 1% to 11% Hemidesmus indicus, about 1% to 11% Acorus calamus, about 1% to 11% Swertia chirata Buch, about 1% to 11% Ptychotis Ajowan, about 1% to 11% Coriander satuvum.

In yet another embodiment, a medicinal composition, P05, is made up of about 1% to 11% Piper longum Linn, about 1% to 11% Terminalia chebula Retz, about 1% to 11% Murraya koenigii, about 1% to 11% Zingiber officinale, about 1% to 11% Glycyrrhiza glabra, about 1% to 11% Curcuma longa, about 1% to 11% Pterocarpus marsupium, about 1% to 11% Alpinia galanga, about 1% to 11% Acorns calamus, about 1% to 11% Asparagus officinalis, about 1% to 11% Camellia sinensis, about 1% to 11% Ocimum sanctum, about 1% to 11% Sesamum indicum, about 1% to 11% Punica granatum.

In yet another embodiment, a medicinal composition, P06, is made up of about 1% to 11% Piper longum, about 1% to 11% Terminalia chebula Retz, about 1% to 11% Murraya koenigii, about 1% to 11% Zingiber officinale, about 1% to 11% Glycyrrhiza glabra, about 1% to 11% Curcuma longa, about 1% to 11% Centella asiatica, about 1% to 11% Cyperus rotundus, about 1% to 11% Terminallia bellerica, about 1% to 11% Hemidesmus indicus, about 1% to 11% Piper nigrum, about 1% to 11% Asparagus racemosus, about 1% to 11% Pterocarpus marsupium, and about 1% to 11% Phyllanthus niruri.

In yet another embodiment, a medicinal composition, P07, is made up of about 1% to 11% Piper longum, about 1% to 11% Terminalia chebula Retz, about 1% to 11% Murraya koenigii, about 1% to 11% Zingiber officinale, about 1% to 11% Glycyrrhiza glabra, about 1% to 11% Curcuma longa, about 1% to 11% Centella asiatica, about 1% to 11% Cyperus rotundus, about 1% to 11% Tinospora cordifolia, about 1% to 11% Hemidesmus indicus, about 1% to 11% Andrographis paniculata, about 1% to 11% Alpinia galanga, about 1% to 11% Boerhavia diffusa, and about 1% to 11% Cassia fistula. More prissily, curcuma longa extract makes 9.1%, Murraya koenigii makes 0.9% and rest of the extract, each of them makes 7.5% of the composition.

The medicinal composition being in edible form, the composition can be administered in capsule, tablet, mini tablet, granule, sachet, powder, paste, ampoule, solution, suspension, emulsion, pills or cream. In some embodiment the composition can be made part of health bar, cookies, powder, daily supplement, emulations, solutions and drinks.

A method to prepare medicinal composition derived from the extracts of about 10 to 14 plant varieties selected from a group of 31 distinct plants and that forms Primary, Secondary and Tertiary constituents in any one of the formulation from P01 to P07.

The ratio in which Primary constituents, Secondary constituents and Tertiary constituents are blended in different embodiments is provided in Table 2a. The ratios provided are different enabling embodiments and do not limit the claims in any ways.

Ratio of primary: secondary: tertiary constituents. *ratio between primary: Secondary constituents.

TABLE 2a

| Composition | Ratios |
|---|---|
| P01 | 2:3:1-4:4:1 |
| P02 | 2:3:1-5:7:2 |
| P03 | 1:2-1:4* |
| P04 | 1:1:1-2:2:1 |
| P05 | 1:1:1-3:2:3 |
| P06 | 5:8:1-4:7:3 |
| P07 | 3:4:1-5:7:2 |

The plants selected for the composition are segregated into different plant parts such as; roots, stems, bark, rhizome, leaves, fruit and seeds. Each plant part is extracted separately, that is, all the roots are extracted together, all the seeds are extracted together, and all the leavers are extracted together and so on. The different extracts are blended together to form a single composition of medicinal composition. More preferably, each plant is extracted separately, their extract is standardised, and such standardised extract are blended to reach the final composition.

The plant parts used from different plant in the medicinal composition is provided in the table below.

TABLE 2b

| | Primary | Secondary | |
|---|---|---|---|
| | Constituents | Constituents | Tertiary Constituents |
| Leaves | Murraya koenigii | Tinospora cordifolia | Boerhavia diffusa |
| | | Hemidesmus indicus | |
| | | Andrographis | |
| | | paniculata | |
| Seeds | | Hemidesmus indicus | Sesamum indicum |
| | | | Punica granatum |
| | | | Ajowa |
| | | | (Ptychotis ajowan) |
| | | | Cassia fistula |
| | | | Phyllanthus niruri |
| | | | Emblica officinalis |
| Fruit | Piper longum Linn | Piper nigrum | Emblica officinalis |
| | Terminalia chebula | Hemidesmus indicus | Cassia fistula |
| | Retz | Terminallia bellerica | Phyllanthus niruri |
| | | Andrographis | |
| | | paniculata | |
| Bark | | Terminallia bellerica | Swertia chirata Buch |
| | | Pterocarpus | Cassia fistula |
| | | marsupium | |
| Stem | | Hemidesmus indicus | Asparagus officinalis |
| | | Andrographis | |
| | | Paniculata | |
| | | Tinospora cordifolia | |
| Rhizome\Roots | Zingiber officinale | Curcuma longa | |
| | | Centella asiatica | |
| | | Cyperus rotundus | |
| | | Alpinia galangal | |
| | Glycyrrhiza glabra | Hemidesmus indicus | |
| | | Asparagus racemosus | |
| | | Acorus calamus | |

The Primary constituents are a mixture of fruit extract of Piper longum Linn, and Terminalia chebula Retz (First extract); rhizomes of Zingiber officinale and roots of Glycyrrhiza glabra (second extract) and leaves of Murraya koenigii (Third extract). The ratio between first extract to second extract to third extract is 7:5:1 to 3:2:1, more preferably about 4:3:1.

The extracts of each constituent is suspended in water and made into a homogenous suspension. The suspension is concentrated and dried to obtain a powdered composition.

The extraction is conducted in a soxhlet extractor. The solvent is selected from a group but not limiting to of polar solvents, methanol, ethanol, ethyl acetate, hydro-alcoholic solvents and hexane. The blended extract can again be extracted to enhance the purity.

The Primary, Secondary and Tertiary constituents are separately extracted from the respective plants source with solvent to obtain a pure extract and then the extracts are blend in the desired ratio provided in P01 to P07.

In some embodiment the selected plant parts constituting the medicinal composition are taken in desired ratio and are pulverized to obtain a homogenized mixture. The homogenized mixture is extracted with solvent repeatedly, the solvent is collected, concentrated and tested for TDS, if the desired TDS is obtained it is sent for drying, the concentrate is continuously recycled to maintain a desired TDS. Lyophilisation can also be used for drying or any other method known in the art.

One embodiment is a product which is designed to deliver a specific quantity of the medicinal composition to a human. The delivery of the medicinal compositions can be done through tablet, capsule, soft gel capsule, powdered sachets, solution, emulsion, cream, past, granules. The medicinal composition can be in a dosage form of about 250 mg to 2000 mg, preferably 500 to 1000 mg and more preferably 800 mg. The medicinal composition can be blended with a pharmaceutically acceptable excipient and made into a dose form. The medicinal composition makes up about 40% to 80% of the blend.

One embodiment provides a method of making medicinal composition with primary constituents. In a conical mixture all the ingredients that are the plant extracts are weighted and fed. The specified quantity for each ingredient is provided in the table 3.

TABLE 3

| | Standardised with | | |
|---|---|---|---|
| | | Percentage | Approximant |
| Extracts | Actives | by weight | Quantity taken |
| Piper longum | Alkaloid | 1-10% | 25 Kg |
| Terminalia chebula | Gallic acid | 1-10% | 25 Kg |
| | Polyphenol | 20-30% | |
| Murraya koenigii | Alkaloid | 1-10% | 3 Kg |
| Zingiber officinale | Total Gingerols | 0.1-10% | 25 Kg |
| Glycyrrhiza glabra | Glycyrrhizic acid | 0.1-10% | 25 Kg |

A homogenised mixture is obtained from the conical mixture and the mixture is a free flowing powder. The free flowing powdered mixture is the medicinal composition.

Piper Longa extract with about 1%-4% of alkaloid can be obtained through solvent extraction of Piper longum fruit. The fruits can be extracted with a hydro alcoholic solvent to obtain the desired extract, a preferred solvent be 70% Ethanol. The Piper Longa extract is a semi-solid extract.

Terminalia chebula extract with about 1%-4% of alkaloid and 1-3% gallic acid can be obtained through solvent extraction of Terminalia chebula fruit. The fruits can be extracted with a hydro alcoholic solvent to obtain the desired extract, a preferred solvent be 70% Ethanol.

Murraya koenigii extract with about 1%-4% of alkaloid can be obtained through solvent extraction of Murraya koenigii leaves. The fruits can be extracted with a hydro alcoholic solvent to obtain the desired extract, a preferred solvent be 70% Ethanol.

Zingiber officinal extract with about 0.1%-2% of alkaloid can be obtained through solvent extraction of Zingiber officinal rhizome. The fruits can be extracted with an alcoholic solvent to obtain the desired extract, a preferred solvent be Ethyl acetate. Preferably the extract is blended with suitable excipient to make the extract in powdered form with desired level of total gingerol.

Glycyrrhiza glabra extract with about 1%-4% of Glycyrrhizic acid can be obtained through solvent extraction of Glycyrrhiza glabra rhizome. The fruits can be extracted with a hydro alcoholic solvent to obtain the desired extract, a preferred solvent be 70% Ethanol.

In another embodiment, a method to prepare a medicinal composition in accordance with the formulations P01 to P07 is provided. For the formulation P07 along with the primary constituents, extracts of Curcuma longa, Centella asiatica, Cyperus rotundus, Tinospora cordifolia, and Hemidesmus indicus from secondary constituent and Alpinia galangal, Boerhavia diffusa, and Boerhavia diffusa from tertiary constituents are also made part of the medicinal composition.

Another embodiment provides a method to inhibit the growth of pathogenic bacteria in gastrointestinal system without disturbing the normal gut micro flora by administering medicinal composition. The medicinal composition is selected from the primary constituents' composition, P01, P02, P03, P04, P05, P06 or P07. The pathogenic bacteria that are inhibited are; Escherichia coli (P1), Staphylococcus aureus (P2), Salmonella typhimurium (P3), Klebsiella oxytoca (P4), Vibrio cholera (P5), Helicobacter pylori (P6), and Shigella dysenteriae (P7). The normal micro flora of the gut that is not harmed by the medicinal composition includes; Lactobacillus acidophilus (BB1), Streptococcus thermophilus (BB2), Bacteroides fragilis (BB3), Clostridium butyricum (BB4), and Faecalibacterium prausnitzii (BB5).

Some embodiments provide a method of preparing a first gastrointestinal health enhancing composition. The method includes collecting fresh rhizomes of Zingiber officinale and roots of Glycyrrhiza glabra. Washing the rhizomes and chopping into flakes, drying the flakes to obtain dried flakes. Extracting dried flakes with 90% methanol and collecting the solvent part of methanol extraction, concentrating the solvent part of the methanol extraction and drying to obtain a powder of an alcoholic extract of Zingiber officinale, and Glycyrrhiza glabra collecting fresh fruits of Piper longum and Terminalia chebula, washing and chopping the fresh fruits into flakes, drying the flakes, extracting the flakes with 90% methanol and collecting the solvent part of the methanol extraction. Filtering and drying the solvent part of the methanol extraction to obtain a powder of an alcoholic extract of fruits of Piper longum and Terminalia chebula.

Collecting fresh leaves of Murraya koenigii, washing, chopping the fresh leaves and drying the chopped leaves to obtain dried flakes. Extracting the dried flakes with 90% methanol and collecting the solvent part of the methanol extraction. Filtering, concentrating and drying the solvent part of the methanol extraction to obtain a powder of an alcoholic extract of leaves of Murraya koenigii.

Blending the powder of the alcoholic extract of Zingiber officinale, and Glycyrrhiza glabra of step a), the powder of powder of the alcoholic extract of fruits of Piper longum and Terminalia chebula of step b) and the powder of the alcoholic extract of leaves of Murraya koenigii to obtain the first gastrointestinal health enhancing composition.

Some embodiments provide a method for preparing second gastrointestinal health enhancing composition. The method includes; collecting fresh rhizomes of Centella asiatica, Curcuma longa, Cyperus rotundus, and Alpinia galangal. Washing the rhizomes and chopping into flakes and drying the flakes to obtain dried flakes. Extracting dried flakes with 90% methanol and collecting the solvent part of methanol extraction, concentrating the solvent part of the methanol extraction and drying to obtain a powder of an alcoholic extract of Centella asiatica, Curcuma longa, Cyperus rotundus, and Alpinia galangal. Collecting fresh stem, bark and leaf of Tinospora cordifolia, Andrographis paniculata, Cassia fistula, Boerhaavia diffusa, and Centella asiatica. Washing the fresh stem, bark and leaf and chopping into flakes and drying the flakes to obtain dried flakes. Extracting dried flakes with 90% methanol and collecting the solvent part of methanol extraction. Concentrating the solvent part of the methanol extraction and drying to obtain a powder of an alcoholic extract of fresh stems, bark and leaves of Tinospora cordifolia, Andrographis paniculata, Cassia fistula, Boerhaavia diffusa, and Centella asiatica. Mixing the powder of the alcoholic extract of Centella asiatica, Curcuma longa, Cyperus rotundus, and Alpinia galangal with the powder of the alcoholic extract of fresh stems, bark and leaves of Tinospora cordifolia, Andrographis paniculata, Cassia fistula, Boerhaavia diffusa, and Centella asiatica to obtain a mixture and Blending the first gastrointestinal health enhancing composition with the mixture to obtain the second gastrointestinal health enhancing composition.

Some embodiments provide a method of preparing a gastrointestinal health enhancing composition. The method includes; collecting a plant part selected from the group consisting of leaf, stem, bark, rhizome, root, fruit and combinations thereof, washing the plant part and chopping into flakes and drying the flakes to obtain dried flakes. Extracting dried flakes with a solvent and collecting the solvent part after extraction. Concentrating the solvent part and drying to obtain a powder of an extract of the plant part. The leaf is selected from the group consisting of Murraya Koenigii, Boerhaavia diffusa, Centella asiatica, Andrographis paniculata, the stem is selected from the group consisting of Tinospora cordifolia, Andrographis paniculata, the bark is Cassia fistula, the rhizome is selected from the group consisting of Hemidesmus indicus, Cyperus rotundus, Alpinia galanga, Curcuma longa, and Zingiber officinale, the root is Glycyrrhiza glabra, wherein the fruit is selected from the group consisting of Piper longum and Terminalia chebula. The solvent selected from the group consisting of hydro alcoholic 70% methanol and ethyl acetate. The powder of the extract can be an extract of the leaf of Murraya koenigii, an extract of the leaf of Boerhaavia diffusa, an extract of the leaf of Centella asiatica, an extract of the leaf of Andrographis paniculata, an extract of the stem of Tinospora cordifolia, an extract of the stem of Andrographis paniculata, an extract of the bark of Cassia fistula, an extract of the rhizome of Hemidesmus indicus, an extract of the rhizome of Cyperus rotundus, an extract of the rhizome of Alpinia galanga, an extract of the rhizome of Curcuma longa, an extract of the rhizome of Zingiber officinale, an extract of the root of Glycyrrhiza glabra, an extract of fruit of Piper longum, or an extract of fruit of Terminalia chebula. A first extract is obtained by combining the extract of leaf of Murraya koenigii, extract of the rhizome of Zingiber officinale, and an extract of the root of Glycyrrhiza glabra, an extract of fruit of Piper longum, and an extract of fruit of Terminalia chebula.

Some embodiments provide a method of preparing a second extract by combining an extract of the leaf of Murraya koenigii, an extract of the leaf of Boerhaavia diffusa, an extract of the leaf of Centella asiatica, an extract of the leaf of Andrographis paniculata, an extract of the stem of Tinospora cordifolia, an extract of the stem of Andrographis paniculata, an extract of the bark of Cassia fistula, an extract of the rhizome of Hemidesmus indicus, an extract of the rhizome of Cyperus rotundus, an extract of the rhizome of Alpinia galanga, an extract of the rhizome of Curcuma longa, an extract of the rhizome of Zingiber officinale, an extract of the root of Glycyrrhiza glabra, an extract of fruit of Piper longum, and an extract of fruit of Terminalia chebula,

Some embodiments provide a method of preparing a third extract by combining the extract of the leaf of Murraya koenigii, an extract of the leaf of Cinderella asiatica, an extract of the leaf of Andrographis paniculata, an extract of the stem of Andrographis paniculata, an extract of the bark of Cassia fistula, an extract of the rhizome of Cyperus rotundus, an extract of the rhizome of Curcuma longa, an extract of the rhizome of Zingiber officinale, an extract of the root of Glycyrrhiza glabra, an extract of fruit of Piper longum, an extract of fruit of Terminalia chebula, an extract of fruit of Terminalia bellerica, an extract of fruit of Momordica charantia, an extract of fruit of Piper nigrum, and an extract of root of Asparagus racemosus.

Another embodiment provides a method of improving gastrointestinal health of a patient by administering medical gastrointestinal health enhancing composition. Improvement in gastrointestinal health is assessed by decrease in abdominal pain, gastric bloating (abdominal distension), nausea/vomiting, eructation, flatus, borborygmus, acid reflux, Tightness in upper abdomen, Heart Burn, pain while passing stools or incomplete evacuation, feelings of incomplete control and inability to control the urgency of passing stools, difficulty in passage of stool, hard stools, and loose stools.

Another embodiment provides a method of improving quality of life by administering the medical gastrointestinal health enhancing composition. The quality of life is assessed based on gastrointestinal symptom rating scale, world health organization quality of life scale and patient assessment of constipation-quality of life scale.

### EXAMPLE 1

### Method of Preparing Primary Constituents' Composition

Raw material was collected, fresh rhizomes of Zingiber officinale (R1), and roots of Glycyrrhiza glabra (R2), were collected in a ratio of 3:2 (total 100 kg).

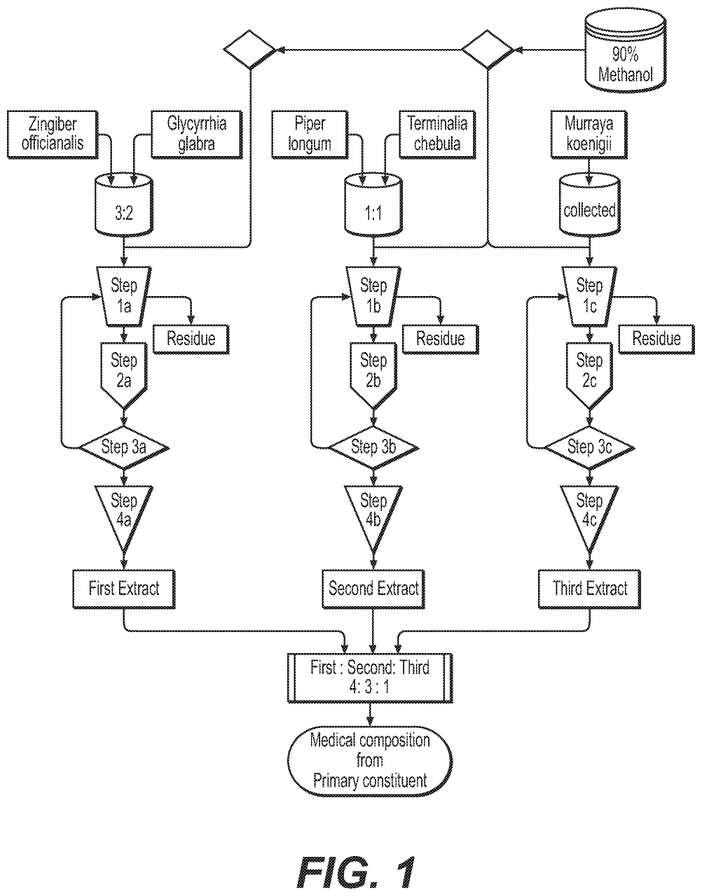

Raw material was washed and chopped into flakes and dried in a hot air oven at around 110° C. for 10 hours. Dried flakes were charged in to an extractor and around 200 litres of 90% methyl alcohol was pumped into the extractor and kept for a contact time of 3 hours (Step 1a) (Refer FIG. 1).

Then the solvent part (methanol part) was collected and fresh methanol was pumped again into the extractor and extraction repeated thrice. All the extracts (methanol part) were pooled, filtered and concentrated in an agitated thin film evaporated (Step 2a).

The total dissolved solids (TDS) was measured continuously and if the TDS was less than desired, the concentrate was recycled back to the reactor till the desired TDS was achieved (Step 3a).

The concentrate was dried in an Agitated thin film drier (ATFD) which was working under vacuum 700 mm Mercury (Step 4a).

Dried product (5 Kg) was discharged from the bottom of the vessel and then pulverized to obtain of powder of an alcoholic extract of Zingiber officinale, and Glycyrrhiza glabra [First extract].

Fresh fruits of Piper longum Linn (R3), and Terminalia chebula Retz (R4) were collected in 1:1 ratio by weight (100 kg). Fresh fruits were washed and chopped into flakes and dried in a hot air oven at around 110° C. for 10 hours.

Dried flakes were charged in to an extractor and around 200 litres of 95% methyl alcohol was pumped into the extractor and kept for a contact time of 3 hours (Step 1b).

Then the solvent part (methanol part) was collected and fresh methyl alcohol pumped again into the extractor and extraction repeated thrice. All the extracts (methanol part) were pooled, filtered and concentrated in an agitated thin film evaporated (Step 2b).

The TDS was measured continuously and if the TDS was less than desired, the concentrate was recycled back to the reactor till the desired TDS was achieved (Step 3b).

The concentrate was dried in an Agitated thin film drier (ATFD) which was working under vacuum 700 mm Mercury (Step 4b).

Dried product (4 Kg) was discharged from the bottom of the vessel and then pulverized to obtain of powder of an alcoholic extract of fruits Piper longum Linn, and Terminalia chebula Retz [Second extract].

Fresh leaves of Murraya koenigii (R5) were collected (100 kg). Fresh leaves were washed and chopped and dried in a hot air oven at around 110° C. for 10 hours.

Dried flakes were charged in to an extractor and around 200 litres of 95% methyl alcohol was pumped into the extractor and kept for a contact time of 3 hours (Step 1c).

Then the solvent part (methanol part) was collected and fresh methyl alcohol pumped again into the extractor and extraction repeated thrice. All the extracts (methanol part) were pooled, filtered and concentrated in an agitated thin film evaporated (Step 2c).

The TDS was measured continuously and if the TDS was less than desired, the concentrate was recycled back to the reactor till the desired TDS is achieved (Step 3c).

The concentrate was dried in an Agitated thin film drier (ATFD) which was working under vacuum 700 mm Mercury (Step 4c).

Dried product (2 Kg) was discharged from the bottom of the vessel and then pulverized to obtain of powder of an alcoholic extract of leaves of Murraya koenigii [Third extract].

The First, second and third extracts are blended in a double cone mixture in a ratio of 4:3:1 to form a Primary constituents' medicinal gastrointestinal health enhancing composition (8 Kg).

### EXAMPLE 2

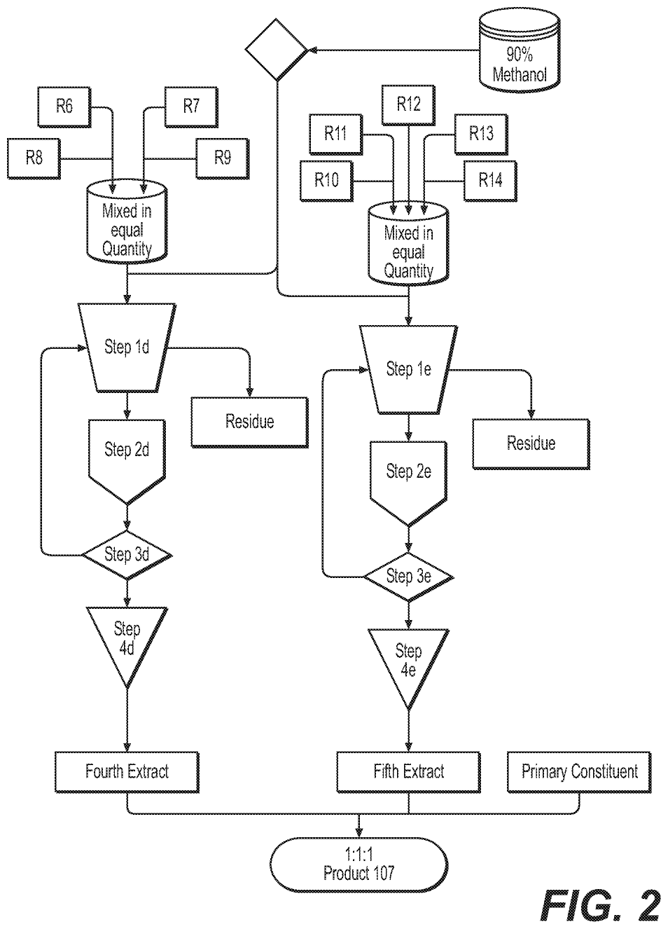

Method to Prepare the Medicinal Composition using Solvent Extraction Process (Refer FIG. 2)

Fresh rhizomes of Centella asiatica (R6), Curcuma Longa (R7), Cyperus rotundus (R8), and Alpinia galangal (R9) are collected in equal quantity, 20 Kg each.

Fresh rhizomes were washed and chopped into flakes and dried in a hot air oven at around 110° C. for 10 hours.

Dried flakes were charged in to an extractor and around 200 litres of 95% methyl alcohol was pumped into the extractor and kept for a contact time of 3 hours (Step 1d). Then the solvent part (methanol part) was collected and fresh methyl alcohol pumped again into the extractor and extraction repeated thrice.

All the extracts (methanol part) were pooled, filtered and concentrated in an agitated thin film evaporated (Step 2d).

The TDS is measured continuously and if the TDS was less than desired the concentrate was recycled back to the reactor till the desired TDS was achieved (Step 3d).

The concentrate was dried in an Agitated thin film drier (ATFD) which was working under vacuum 700 mm Mercury (Step 4d), an extract was obtained [Fourth Extract].

Fresh stems, bark and leaves of Tinospora cordifolia (R10), Andrographis paniculata (R11), Cassia fistula (R12), Boerhaavia diffusa (R13), and Centella asiatica (R14) are collected 15 Kg each.

Fresh leaves, bark and stems were washed and chopped and dried in a hot air oven at around 110° C. for 10 hours. Dried flakes were charged in to an extractor and around 200 litres of 95% methyl alcohol was pumped into the extractor and kept for a contact time of 3 hours (Step 1e).

Then the solvent part (methanol part) was collected and fresh methyl alcohol pumped again into the extractor and extraction repeated thrice.

All the extracts (methanol part) were pooled, filtered and concentrated in an agitated thin film evaporated (Step 2e).

The TDS is measured continuously and if the TDS was less than desired, the concentrate was recycled back to the reactor till the desired TDS was achieved (Step 3e).

The concentrate was dried in an Agitated thin film drier (ATFD) which was working under vacuum 700 mm Mercury (Step 4e).

Dried product (2 Kg) was discharged from the bottom of the vessel and then pulverized to obtain of powder of an alcoholic extract [Fifth Extract].

The fourth extract and the fifth extract are mixed together and the mixture was blended with Primary constituents' composition from example 1 in a double cone mixture in a ratio Primary constituents' composition to the mix 1:2 ratio.

The final homogenized medicinal composition obtained was P07. Similar medicinal composition such as P01, P02, P03, P04, P05 or P06 can also be prepared by the above said method.

### EXAMPLE 3

### In-Vitro Anti-Bacterial Screening

The purpose of this illustration was to show the activity of various medicinal compositions against the Standard. The illustration in no way limits the scope of the disclosed teachings to the blends tested in this example.

Anti-bacterial activity of medicinal composition was assessed in-vitro against, Escherichia coli (P1), Staphylococcus aureus (P2), Salmonella typhimurium (P3), Klebsiella oxytoca (P4), Vibrio cholera (P5), Helicobacter pylori (P6), and Shigella dysenteriae (P7). Activity was also assessed against beneficial microbes such as; Lactobacillus acidophilus (BB1), Streptococcus thermophilus (BB2), Bacteroides fragilis (BB3), Clostridium butyricum (BB4) and Faecalibacterium prausnitzii (BB5).

Faecali-bacterium prausnitzii by resazurin dye reduction method (Sarker S D, Nahar L, Kumarasamy Y. Microtitre plate-based antibacterial assay incorporating resazurin as an indicator of cell growth, and its application in the in-vitro antibacterial screening of phytochemicals, Methods. 2007 August; 42(4): 321-324).

The sample selected for study are medicinal compositions P07, P05, P03 and P02, Primary Constituents' composition (PC), along with Piper longum Linn (PL), Terminalia chebula (TC), Murraya koenigii (MK), Zingiber officinale (ZO), and Glycyrrhiza glabra (GG). The activities of samples are compared in this study.

Each sample was tested separately against the selected bacteria strains to determine their Minimum Inhibitory Concentration for each bacteria strain. A sterile 96 well (8×12) plate was prepared and labeled under aseptic conditions. Stock solution of test sample was prepared by dissolving 100 mg powdered sample in DMSO to get concentration 10 mg/ml.

A volume of 100 μl of test sample solution was pipette into the first row of the plate. To all other wells 50 μl of nutrient broth was added.

Serial dilutions were performed using a multichannel pipette to get final concentrations from 1000 μg/ml to 7.81 μg/ml i.e. (1000, 500, 250, 125, 62.5, 31.25, 15.625 and 7. 8125 μg/ml).

To each well 10 μl of resazurin indicator solution and 30 μl of 3× strength isosensitized broth was added. Finally, 10 μl of microbial suspension (5×10⁶ cfu/mL) was added to each well to achieve a concentration of 5×10⁵ cfu/mL.

Each plate was wrapped loosely with cling film to ensure that cultures did not become dehydrated. Each Plate had a set of positive (with organism), negative (without organism) and a standard (ciprofloxacin).

The plates were prepared and placed in an incubator at 37° C. for 18-24 h. The colour change was then assessed visually. Any colour changes from purple to pink or colourless were recorded as positive for bacterial growth. The lowest concentration at which no colour change occur (absence of growth) was taken as the MIC (Minimum Inhibitory Concentration) value.

Importantly, the significant antibacterial activity was exhibited against unfavorable bacteria with lower MIC values (15.625-250 μg/ml) in contrast to antibacterial activity observed against favorable bacteria with higher MIC values (250-1000 μg/ml).

MIC of various medicinal compositions and standard (ciprofloxacin) against bacteria by micro-titre method.

TABLE 4

| | | MIC value (μg/ml) | | | | | |
|---|---|---|---|---|---|---|---|
| | | | | | | | Cipro- |
| ORGANISM | | P02 | P03 | P05 | P07 | PC | floxacin |
| Pathogens | P1 | 250 | 250 | 500 | 125 | 250 | 1000 |
| | P2 | 250 | 500 | 500 | 125 | 250 | 500 |
| | P3 | 125 | 250 | 500 | 125 | 1000 | 1000 |
| | P4 | 125 | 125 | 250 | 125 | 250 | 500 |
| | P5 | 62.5 | 62.5 | 125 | 31.25 | 125 | 125 |
| | P6 | 31.25 | 15.625 | 62.5 | 15.625 | 62.5 | 15.125 |
| | P7 | 125 | 125 | 250 | 125 | 250 | 15.125 |
| Beneficial | BB1 | 500 | 1000 | 500 | 1000 | 500 | 250 |
| bacteria | BB2 | >1000 | >1000 | >1000 | 1000 | >1000 | 31.25 |
| | BB3 | 500 | >1000 | >1000 | 1000 | 1000 | 62.5 |
| | BB4 | 1000 | 1000 | >1000 | 500 | >1000 | 125 |
| | BB5 | 500 | 1000 | 1000 | 500 | >1000 | 31.25 |

Primary constituents were tested individually and compared with Primary constituents' composition from example 1. MIC of one constituent was quite different from other constituent. Some constituents were good at reducing the pathogenic bacteria at lower dose than other, so could not inhibit the growth at all. Some constituents inhibited the growth of favorable and pathogenic strains at similar MIC.

MIC of individual constituents of primary constituent and primary constituent (as prepared in example 1) (ciprofloxacin) against bacteria by micro-titre method.

TABLE 5

| | | MIC value (μg/ml) | | | | | |
|---|---|---|---|---|---|---|---|
| ORGANISM | | R1 | R2 | R3 | R4 | R5 | PC |
| Patho- | P1 | 500 | 1000 | 1000 | 500 | 125 | 500 |
| gens | P2 | 500 | 500 | 500 | 1000 | 125 | 250 |
| | P3 | 250 | 1000 | 1000 | >1000 | 1000 | 1000 |
| | P4 | 125 | 1000 | 250 | 1000 | 1000 | 250 |
| | P5 | 125 | 500 | 1000 | >1000 | 1000 | 250 |
| | P6 | 62.5 | 1000 | 250 | 125 | 500 | 125 |
| | P7 | 31.25 | 500 | 250 | 31.25 | 500 | 62.5 |
| Bene- | BB1 | 1000 | 1000 | 1000 | 1000 | 125 | 500 |
| ficial | BB2 | 1000 | >1000 | >1000 | 1000 | 125 | >1000 |
| bacteria | BB3 | 1000 | 1000 | 500 | 125 | 500 | 1000 |
| | BB4 | 1000 | 1000 | >1000 | 125 | 500 | >1000 |
| | BB5 | 500 | >1000 | 1000 | 250 | 500 | >1000 |

Medicinal compositions; P02, P03, P05, and P07 had a similar outline on antibacterial activity. The MIC of medicinal compositions against Shigella dysentriae, Klebseill aoxytoca, Vibrio cholera, Escherichia coli, Staphylococcus aureus, Salmonella typhimurium was noted to be below 500 μg/ml, in most cases bellow 250 μg/ml, which is 2 to 4 times lower that the standard (ciprofloxacin). At a lower concentration the medicinal compositions were able to inhibit pathogenic bacteria strains and at the same concentration the effect on same favorable bacteria strains such as Lactobacillus acidophilus, Streptococcus thermophilus, Bacteroides fragilis, and Clostridium butyricum were negligible.

The composition of Primary constituents showed similar effect in comparison the medicinal compositions used. The Primary constituents' composition showed significant antibacterial activity against unfavourable bacteria at lower concentration at the same time no adverse activity was observed against favourable bacteria such as Lactobacillus acidophilus, Streptococcus thermophilus, Bacteroides fragilis, and Clostridium butyricum.

### EXAMPLE 4

Human Population Study for Gastrointestianl Relife

The study was a randomized, double blind, placebo controlled, uni-canter, human study to evaluate the efficacy and tolerability of various medicinal compositions on gastrointestinal health and symptoms. There are total four visits and duration of the study is 28 days.

A total of 72 patients (male and female) were enrolled in this clinical trial. Healthy adult men or women between 18-65 years of age and 20-30 kg/m² BMI with self-described unsatisfactory bowel habit, and GSRS (Gastrointestinal Symptom Rating Scale) with every question less than 3 points were included in the study.

Subjects willing to discontinue fibre supplementation and other probiotics, prebiotics, fermented milk, yoghurt or laxatives at least 4 weeks prior to and during the study phase were included. Female subjects of child-bearing potential must be willing to use a reliable method of contraception throughout the study period or those who are surgically sterile.

The enrolled patients were assigned six study groups medicinal composition; P02, P03, P05, P07 and Primary Constituents', along with Placebo. Each subject was given 800 mg of the test sample, an hour before bedtime. Patients were asked to use the medication daily for 28 days with follow-up visit at 2 and 4 weeks.

In study period, there were 3 visits including the screening visit.

In visit 1 (day −5 to −1) Demographic data, medical history, physical and systemic examination, vital parameters including respiratory rate, ECG, chest x-ray, haematology, serology, biochemistry and urine analysis were performed.

On visit 2 (day 0) the eligible subjects were enrolled into the trial and investigational products were given to the patient as per randomization, and he/she started to take sample the next day (day 1).

In visit 3 (day 14), subject satisfaction, stool frequency, stool consistency, ease of passage, WHO-QOL, PAC-QOL, test substance tolerability, adverse events (AE) assessment were performed.

In visit 4 (day 28), subject satisfaction, Fecal microbiota, stool frequency, stool consistency, ease of passage, GSRS (Gastrointestinal Symptom Rating Scale) assessment, WHO-QOL, PAC-QOL, test substance tolerability and adverse events (AE) assessment were done and the unused samples were collected.

### GSRS (Gastrointestinal Symptom Rating Scale)

GSRS was analysed by subjects' self-report improvement of gastrointestinal symptoms across four weeks. The GSRS covers 15 gastrointestinal symptoms and uses a 3-point Likert scale to rate each symptom, depending on how inconvenient it had been during the previous week. A higher score indicates more inconvenient symptoms. Combination scores among 15 questions were assessed in the following five domains: 1. abdominal pain (stomach ache, gastric hunger pains and nausea). 2. Constipation syndrome (constipation, hard stools and feeling of incomplete evacuation). 3. Diarrhea syndrome (diarrhea, loose stools and urgent need to defecate). 4. Indigestion syndrome (gastric borborygmus, gastric bloating, eructation and increased flatus) and 5. Reflux syndrome (heartburn and acid regurgitation).

GSRS was analysed by subjects' self-report improvement of gastrointestinal symptoms across four weeks. The GSRS covers 15 gastrointestinal symptoms and uses a 3-point Likert scale where 1 represents absence of troublesome symptoms and 3 represents very troublesome symptoms, depending on how inconvenient it had been during the previous week. Combination scores among 15 questions were assessed in the following five domains:
1. Abdominal pain (stomach ache, gastric hunger pains and nausea).
2. Constipation syndrome (constipation, hard stools and feeling of incomplete evacuation).
3. Diarrhea syndrome (diarrhea, loose stools and urgent need to defecate).
4. Indigestion syndrome (gastric borborygmus, gastric bloating, eructation and increased flatus) and
5. Reflux syndrome (heartburn and acid regurgitation).

The mean average data for GSRS is presented in a simplified form in Table 5a and 5b.

Consolidated data from the gastrointestinal symptom rating scale (GSRS) for medicinal compositions (P02 P03 and primary constituents' blend) was recorded and provided below.

TABLE 6a

| | | medicinal composition | | | | | |
|---|---|---|---|---|---|---|---|
| Domains | | P02 | | P03 | | PC | |
| Abdominal | Stomach ache | 74% | 69% | 75% | 70% | 69% | 62% |
| pain | Gastric hunger | 60% | | 59% | | 55% | |
| | pains | | | | | | |
| | Nausea | 67% | | 69% | | 62% | |
| Constipation | Constipation | 25% | 47% | 26% | 48% | 16% | 41% |
| syndrome | hard stools and | 51% | | 53% | | 45% | |
| | Incomplete | | | | | | |
| | evacuation | 66% | | 64% | | 61% | |
| Diarrhea | Diarrhea | 48% | 42% | 48% | 42% | 42% | 33% |
| syndrome | Loose stools | 28% | | 27% | | 18% | |
| | Urgent need to | 52% | | 51% | | 41% | |
| | defecate | | | | | | |
| Indigestion | gastric | 57% | | 57% | | 51% | |
| syndrome | borbory-gmus | | | | | | |
| | gastric bloating | 64% | 63% | 63% | 63% | 58% | 57% |
| | eructation | 65% | | 65% | | 57% | |
| | increased flatus | 67% | | 66% | | 60% | |
| Reflux | heartburn | 75% | | 74% | | 70% | |
| syndrome | acid | 67% | 71% | 66% | 70% | 65% | 67% |
| | regurgitation | | | | | | |

The data provided in the table is a compilation of mean score in each group and their percentage improvement over 28 days when compared to placebo, improvement in specific illness associated with larger domain was also listed in the table.

Consolidated data from the gastrointestinal symptom rating scale (GSRS) data for medicinal composition (P07 and P05) and placebo is provided below.

TABLE 6b

| | | medicinal composition | | | | | |
|---|---|---|---|---|---|---|---|
| Domains | | P07 | | P05 | | Placebo | |
| Abdominal | Stomach ache | 74% | 69% | 74% | 68% | 2% | 3% |
| pain | Gastric hunger | 61% | | 60% | | 5% | |
| | pains | | | | | | |
| | Nausea | 68% | | 68% | | 2% | |
| Constipation | Constipation | 25% | 47% | 24% | 47% | 1% | 2% |
| syndrome | hard stools and | 51% | | 50% | | 3% | |
| | Incomplete | 66% | | 67% | | 2% | |
| | evacuation | | | | | | |
| Diarrhea | Diarrhea | 48% | 42% | 50% | 44% | 1% | −0.4% |
| syndrome | Loose stools | 28% | | 29% | | 2% | |
| | Urgent need to | 52% | | 52% | | −5% | |
| | defecate | | | | | | |
| Indigestion | gastric | 57% | 63% | 58% | 64% | −5% | 0.09% |
| syndrome | borborygmus | | | | | | |
| | gastric bloating | 64% | | 65% | | 3% | |
| | eructation | 65% | | 66% | | 2% | |
| | increased flatus | 67% | | 68% | | 0.08% | |
| Reflux | heartburn | 75% | 71% | 77% | 73% | 2% | 3% |
| syndrome | acid regurgitation | 67% | | 69% | | 5% | |

It was observed that in the medicinal composition groups, in all four of them, the proportion of subjects experienced severity of uneasiness has come down significantly when compared to placebo. Among the medicinal composition groups' subjects reported over 60 percent improvement for abdominal pain compared to placebo. Improvement in Constipation and Diarrhea syndrome is over 40% among the medicinal composition groups. About 60% improvement is observed in digestion among medicinal composition groups and about 70% improvement in reflex syndrome.

In the medicinal composition groups i.e.P02, P03, P05 and P07, at the end of study, abdominal pain had about 65-75% decrease, gastric bloating (abdominal distension) had about 55-64% decrease, nausea/vomiting had about 58-67% decrease, eructation had about 55-65% decrease, flatus had about 55-67% decrease, borborygmus about 45-57% decrease, acid reflux about 55-67% decrease, Tightness in upper abdomen about 55-64% decrease, Heart Burn about 55-75% decrease, pain while passing stools or incomplete evacuation had a about 55-66% decrease, feelings of incomplete control and inability to control the urgency of passing stools had about 40-52% decrease, decrease in passage of stool had about 15-25% decrease, hard stools had about 40-51% decrease, increased passage of stools has about 20-28% decrease, and loose stools has about 38-48% decrease.

In the placebo group, at the end of study, abdominal distension had about 2% decrease, nausea/vomiting had about 12% decrease, eructation had no significant decrease, flatus had about 4% decrease, borborygmus about 5% increase, acid reflux about 4% decrease, Tightness in upper abdomen no significant decrease, Heart Burn 2% decrease, pain while passing stools or incomplete evacuation had about 2% decrease, feelings of incomplete control and inability to control the urgency of passing stools had no significant decrease, decrease in passage of stool had about 13% decrease, hard stools had about 2% increase, increased passage of stools had about 2% decrease, and loose stools had about 2% increase.

World Health Organization Quality of Life (WHOQOL)

The WHOQOL-100 quality of life assessment was developed by the WHOQOL Group with fifteen international field centres, simultaneously, in an attempt to develop a quality of life assessment that would be applicable cross-culturally. WHOQOL-BREF (an abbreviated version of the WHOQOL-100) contains a total of 26 questions. To provide a broad and comprehensive assessment, one item from each of the 24 facets contained in the WHOQOL-100 has been included. In addition, two items from the Overall quality of Life and General Health facet have been included.

WHOQOL was analysed by subjects' self-report improvement of perceived quality of life at 0, and 28 days. The WHOQOL-BREF assesses four domains of quality of life: physical health, psychological, social relationships and the environment.

Four domains of quality of life i.e. physical health, psychological, social relationships and the environment were assessed by WHOQOL-BREF. The four domain scores denote an individual's perception of quality of life in each particular domain. The raw scores were converted into a transformed scores (on a 0-100 scale) using tables for each domain.

The data for WHOQOL is presented in the Table 6. There was significant improvement in Physical and Psychological score among subjects from medicinal composition and Primary constituents' group when compared to placebo, not much difference is seen in Secondary and Tertiary constituents group with respective baseline scores.

Percentage improvement in each domain of WHOQOL-BREF from day zero to day 28, for each medicinal composition is compared with placebo.

TABLE 7

| Study | Percentage improvement in QOL | | | | |
|---|---|---|---|---|---|
| Group | Environmental | Social | Physical | Psychological | Overall |
| P07 | 92% | 39% | 79% | 96% | 82% |
| P02 | 91% | 38% | 78% | 94% | 80% |
| P03 | 91% | 40% | 77% | 97% | 82% |
| P05 | 90% | 39% | 80% | 93% | 80% |
| PC | 87% | 35% | 69% | 91% | 73% |
| Placebo | 3.58% | 5.27% | 3.67% | 4.98% | 3.19% |

In the medicinal composition groups of P02, P03, P05P07 and Primary constituents' composition it was found that the Standardized Response Mean (SRM) has a large clinical significance as compared to placebo.

In the overall score domain there was an increase of about 70%-85% in the total score of QOL of medication group as compared to 3.19% in placebo group.

In the domain environment, there was about 80-95% increase in the total score of medication group as compared to 3.58% in placebo.

In the social domain there was a 30%-40% increase in the total score of medication group as compared to 5.27% in placebo.

In the physical domain there was an increase of 65%-80% in the total score of medication group as compared to 3.67% in placebo group.

In the psychological domain there was an increase of 85%-96% in the total score of QOL of medication group as compared to 4.98% in placebo group.

Patient Assessment of Constipation-Quality of Life (PAC-QOL).

PAC-QOL is a self-reported questionnaire, was used to measure the quality of life of patients at 0 and 28 days. The validated PAC-QOL is composed of 26 items grouped into four subscales: physical discomfort, psychosocial discomfort, worries and concerns, and satisfaction.

PAC-QOL scoring was done on a Likert 5-point scale from 0 (Nothing/Never) to 4 (Extremely/Always) in which a lower score reflected a better quality of life.

In case of subgroup satisfaction 0 represent “not at all” and 4 “Extremely”, that means a higher score reflects a better quality of life.

The 26 items of questionnaire were grouped into four subscales: physical discomfort, psychosocial discomfort, satisfaction and worries & concerns. The data is presented in Table 7.

In physical discomfort domain, there was significant difference in medicinal composition groups whereas in the placebo group, difference between days 0 to day 28 was significant.

In psychosocial discomfort, both medicinal composition groups and placebo group had the final score less and both the group had the same statistically significant benefit.

Clinically important changes were for only the medicinal composition groups. The change in the placebo group was clinically unimportant.

In the satisfaction domain, there was significant reduction in score for medicinal compositions as well as placebo group but clinically significant benefits were only in the medicinal composition groups. Similarly, there was statistically significant reduction in the score for worries & concerns in medicinal composition as well as placebo group but clinically significant improvement was in the medicinal composition group only.

PAC-QOL data for different medicinal compositions and placebo groups, increase (+) and decries (−) in percentage of score is compiled in the table.

TABLE 8

| | Percentage change in PAC-QOL Score | | | | People |
|---|---|---|---|---|---|
| | Physical | Physco- | | | with |
| Study | Dis- | Social | | Worries & | clinically |
| Group | comfort | Discomfort | Satisfaction | Concerns | benefit |
| P07 | −66% | −72% | +63% | −69% | 96% |
| P02 | −65% | −73% | +62% | −70% | 96% |
| P03 | −67% | −73% | +64% | −69% | 96% |
| P05 | −67% | −71% | +61% | −65% | 94% |
| PC | −57% | −66% | +57% | −60% | 98% |
| Placebo | +8% | +0.5% | +11% | −6% | 2% |

In the physical discomfort domain there was a 55%-70% decrease in the physical discomfort score in the medication group while there was an increase of 8.46% in the placebo group.

In the psycho-social discomfort domain there was a 65%-75% decrease in the Psycho-social discomfort score in the medication group while there was an increase of 0.46% in placebo group.

In the domain satisfaction, there was about 55%-65% increase in the satisfaction score in the medication group while there was an increase of 11.3% in placebo group.

In the domain worries and concerns there was about 60%-70% decrease in the satisfaction score in the medication group while there was a decrease of 6.52% in placebo group. It was seen that in the medicinal composition groups about 94%-98% people got clinically effective benefit while only 1.9% people got clinically effective benefit in the placebo group.

### EXAMPLE 5

Method to Make P07 and Primary Constituent Medicinal Composition with Individual Extracts

Various parts of fourteen plants are used as raw material. All the required plant parts are separately collected, sorted and prepared for solvent extraction. Detail about the raw material and their designated solvents.

TABLE 9

| | Plant part used | Quantity | |
|---|---|---|---|
| Herb | (Raw Material) | (Kg) | Solvent Used |
| Murraya Koenigii | Leaves | 50 Kg | 70% |
| | | | Methanol |
| Tinospora | Stem | 50 Kg | 70% |
| cordifolia | | | Methanol |
| Glycyrrhiza glabra | Rhizomes | 50 Kg | 70% |
| | | | Methanol |
| Hemidesmus | Rhizomes | 50 Kg | 70% |
| indicus | | | Methanol |
| Cassia fistula | Dried Bark | 50 Kg | 70% |
| | | | Methanol |
| Piper longum | Fruits | 50 Kg | 70% |
| | | | Methanol |
| Cyperus rotundus | Rhizomes | 50 Kg | 70% |
| | | | Methanol |
| Boerhaavia diffusa | Leaves | 50 Kg | 70% |
| | | | Methanol |
| Alpinia galanga | Rhizomes | 50 Kg | 70% |
| | | | Methanol |
| Terminalia chebula | Fruits | 50 Kg | 70% |
| | | | Methanol |
| Centella Asiatica | Leaves | 50 Kg | 70% |
| | | | Methanol |
| Curcuma Longa | Rhizomes | 50 Kg | Ethyl acetate |
| Andrographis | Dried stem & | 50 Kg | 70% |
| paniculata | Leaves | | Methanol |
| Zingiber officinale | Rhizomes | 50 Kg | Ethyl acetate |

Raw material is washed and chopped into flakes and dried in a hot air oven at around 110° C. for 10 hours. Dried flakes were charged in to an extractor and around 150-250 litres of solvent was pumped into the extractor and kept for a contact time of 3 hours. Each raw material was extracted separately by its designated solvent as provided in the table above.

Then the solvent part was collected and fresh solvent was pumped again into the extractor and extraction repeated up to thrice. The TDS is measured continuously once the desired TDS was achieved all the extracts (solvent part) were pooled, filtered and concentrated in an agitated thin film evaporated.

The concentrate was dried in an Agitated thin film drier (ATFD) which was working under vacuum 700 mm Mercury.

For each of the fourteen plant material an extract is obtained, the yield for which varied from 10 to 20%. The final extract in each case was mostly in dry solid form. Each extract from the each of the raw material was then powdered to a 13 mesh size.

### Making Primary Constituents' Composition:

In to 50 Litter double cone mixture 5 Kg each of Zingiber officinale, Glycyrrhiza glabra, Piper longum, and Terminalia chebula and 3 Kg of Murraya koenigii extract was added mixed well. It was mixed till a homogenised mixture is not obtained.

### Making P07 Composition:

In to 50 Litter double cone mixture 5 Kg each of Tinospora cordifolia, Glycyrrhiza glabra, Hemidesmus indicus, Cassia fistula, Piper longum, Cyperus rotundus, Boerhaavia diffusa, Alpinia galanga, Terminalia chebula, Centella Asiatica, Curcuma Longa, Andrographis paniculata, and Zingiber officinale. Along with 3 Kg of Murraya Koenigii extract was added and mixed well to obtain a homogenised composition P07.

The above method can be used by a person skilled in the art to make any of the P01 to P08 composition.

### EXAMPLE 6

### In-Vitro Anti-Bacterial Screening—Determining Zone of Inhibition (ZOI).

Antimicrobial study of each of the extract used in the medicinal composition and it was compared with the actual composition. The ZOI study was conducted for each individual extract.

Sample preparation: The test substance, plant extract 100 mg was dissolved in 1 ml of 20% Dimethyl sufoxide. Dimethyl sufoxide 20% was used as negative control and Vancomycin at 3 mg in 1 ml of 20% Dimethyl sufoxide was used as positive control.

The antimicrobials present in the plant extracts were allowed to diffuse out into the medium and interact in a plate freshly seeded with the test organisms. The resulting zone of inhibition will be uniformly circular as there will be a confluent lawn of growth. The diameter of ZOI can be measured in millimetres.

Petri plates containing 20 ml Muller Hinton medium were seeded with a 24 hr culture of the bacterial strain. 5 mm diameter wells were cut and 100 μl of the plant extract; negative control and positive control were added. The plates were then incubated at 37° C. for 24 h. The antibacterial activity was assayed by measuring the diameter of the inhibition zone.

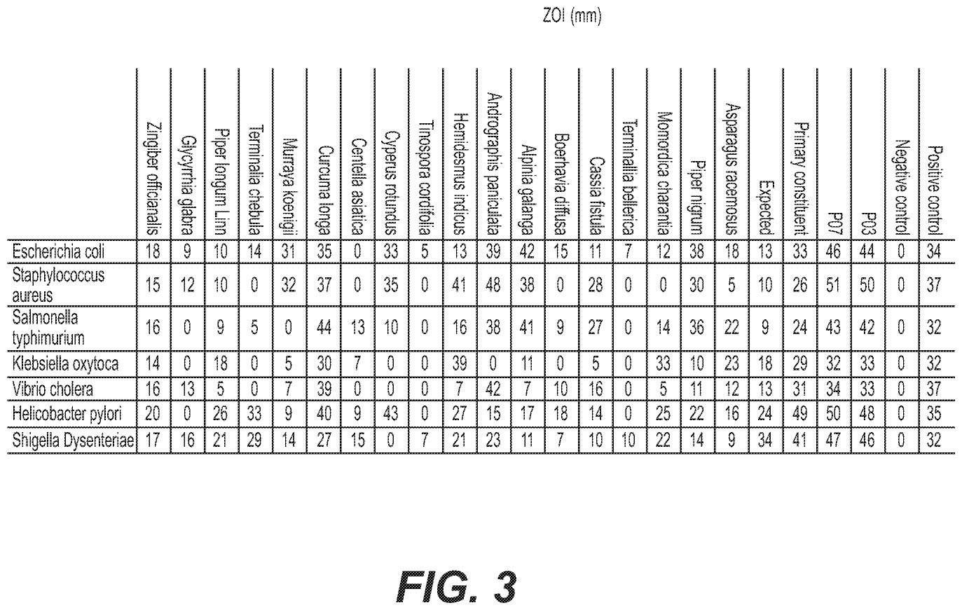

The ZOI for each plant extract is shown in FIG. 3. The ZOI for the primary constituents', P03 and P07 composition made as per example 5 is provided. Primary constituents' composition comprises of Piper longum Linn, Terminalia chebula, Murraya koenigii, Zingiber officinale and Glycyrrhiza glabra.

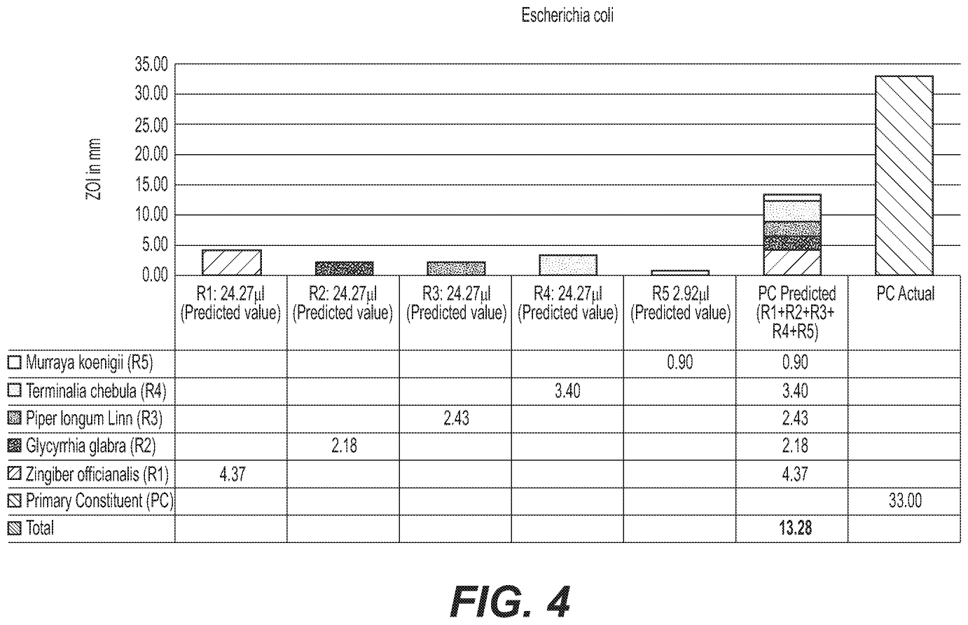

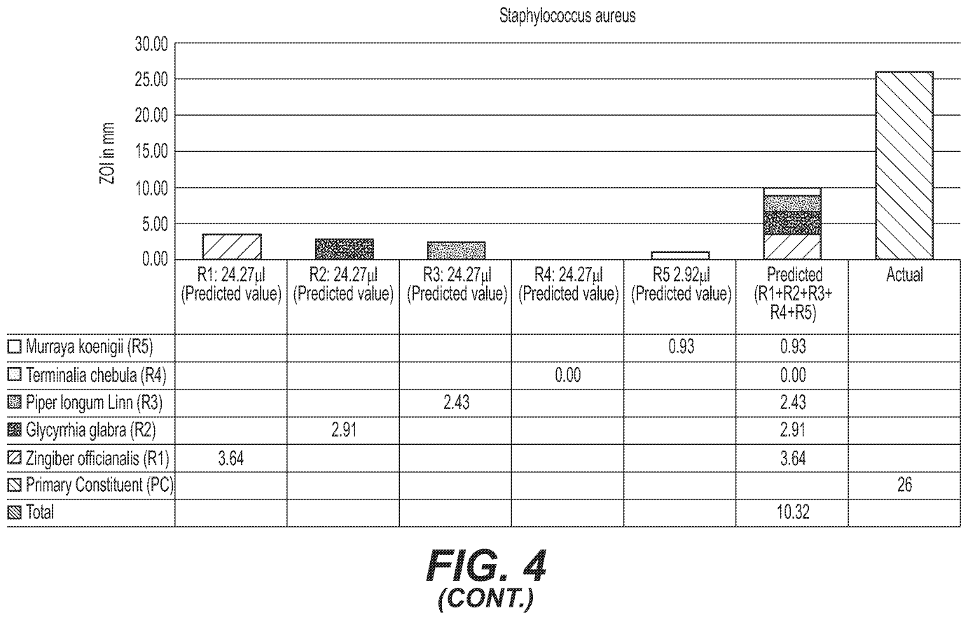

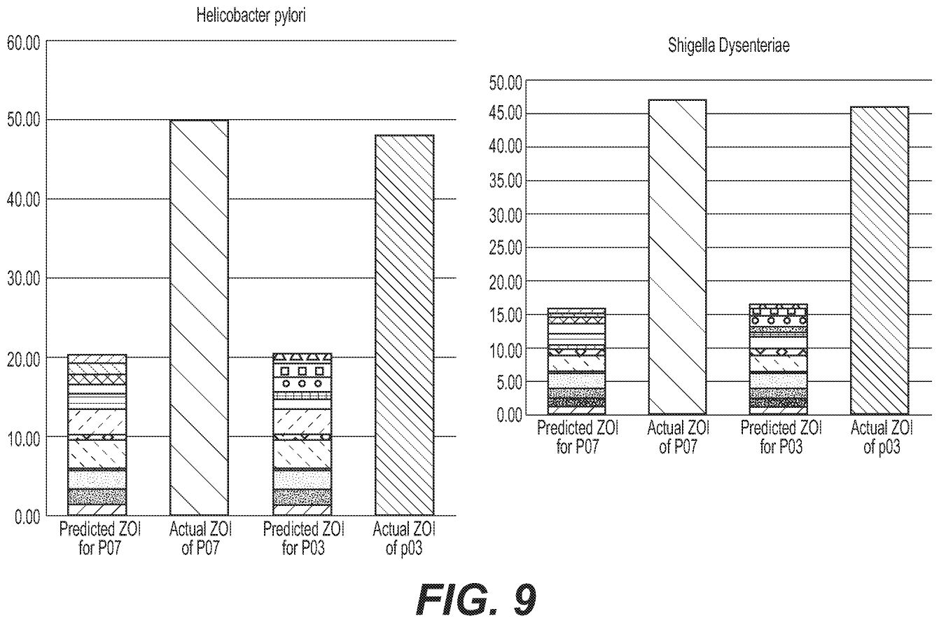

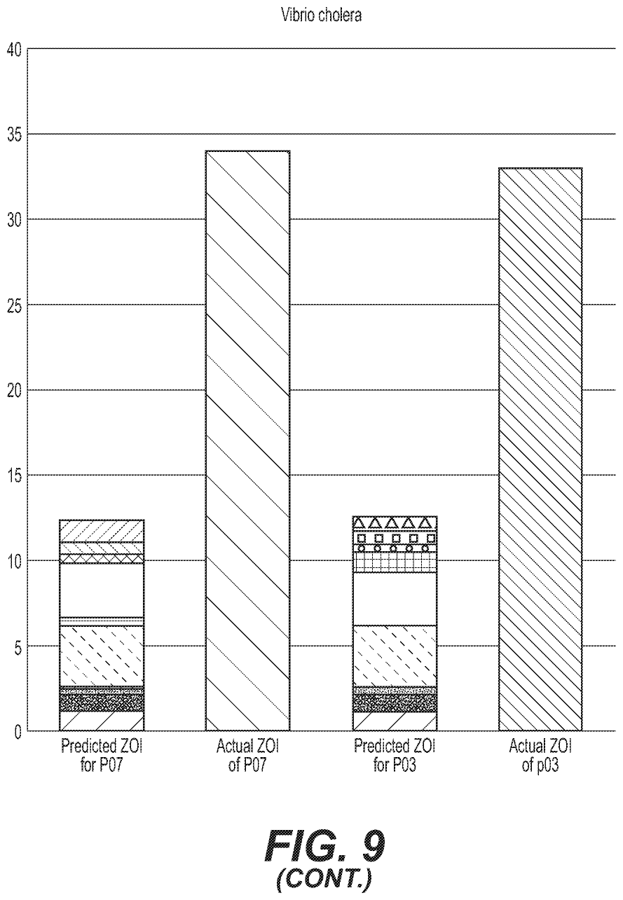

It can be observed from FIG. 4 and FIG. 9 the ZOI of inhibition for primary constituents' composition, P03 and P07 in actual was much higher than the predicted theoretical value. The predicted ZOI was calculated from the data provided in FIG.3 for each plant extract.

A ZOI of 18 mm was observed for 100 μl extract of Zingiber officinale. A lower dose of extract was expected to have a smaller ZOI. Therefore, we assumed that the dosage of extract would be directly proportional to ZOI, that is, the relationship between dosage and ZOI would be a linear function.

A 100 μl of a primary constituents' composition had 24.27 μl extract each of Zingiber officinale, Glycyrrhiza glabra, Piper longum Linn and Terminalia chebula, and 2.92 μl of Murraya koenigii.

Since 100 μl of Zingiber officinale showed a ZOI of 18 mm against Escherichia coli, therefore 24.27 μl was expected to provide a predicted ZOI of 4.37 mm. 18×24.27/100=4.37 mm. Similarly predicted ZOI for Glycyrrhiza glabra was 2.18 mm, Piper longum was 2.43 mm, Terminalia chebula was 3.4 mm, and Murraya koenigii was 0.9 mm. A total predicted ZOI of the primary constituents having extracts of Zingiber officinale, Glycyrrhiza glabra, Piper longum Linn and Terminalia chebula and Murraya koenigii was obtained by adding the predicted ZOI of individual extracts, namely, 4.37 mm+2.18 mm+2.43 mm+3.4 mm+0.9 mm=13.28 mm (FIG. 4). Therefore, the primary constituents' composition was expected to show a ZOI of 13.28 mm based on the stoichiometric amounts of each of the individual components of the extract. However, the observed ZOI for primary constituents' composition was 33 mm.

Therefore, the combination of the primary constituents resulted in a synergistic effect because the observed ZOI was significantly higher than the ZOI predicted based on the stoichiometric amounts of the extracts.

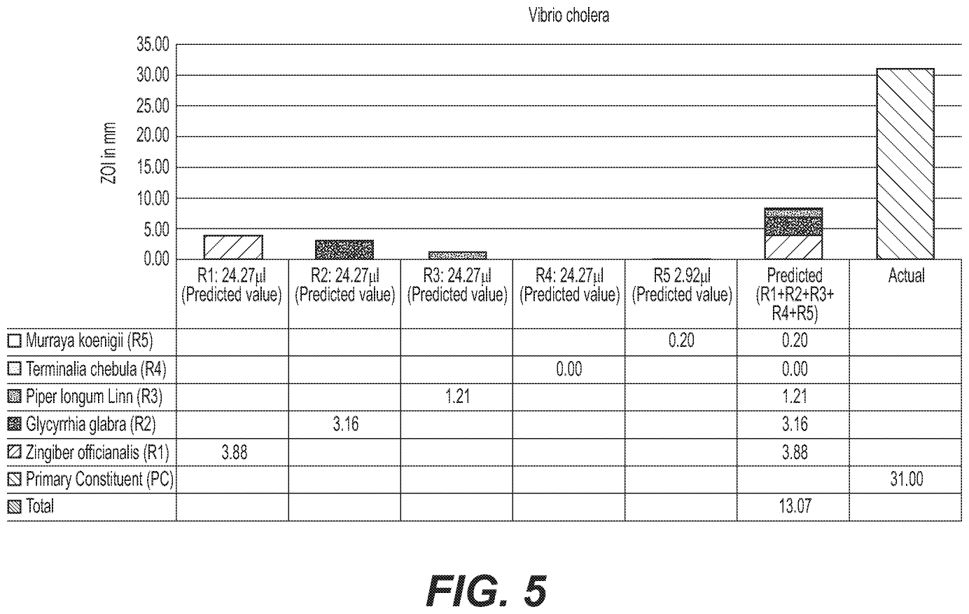

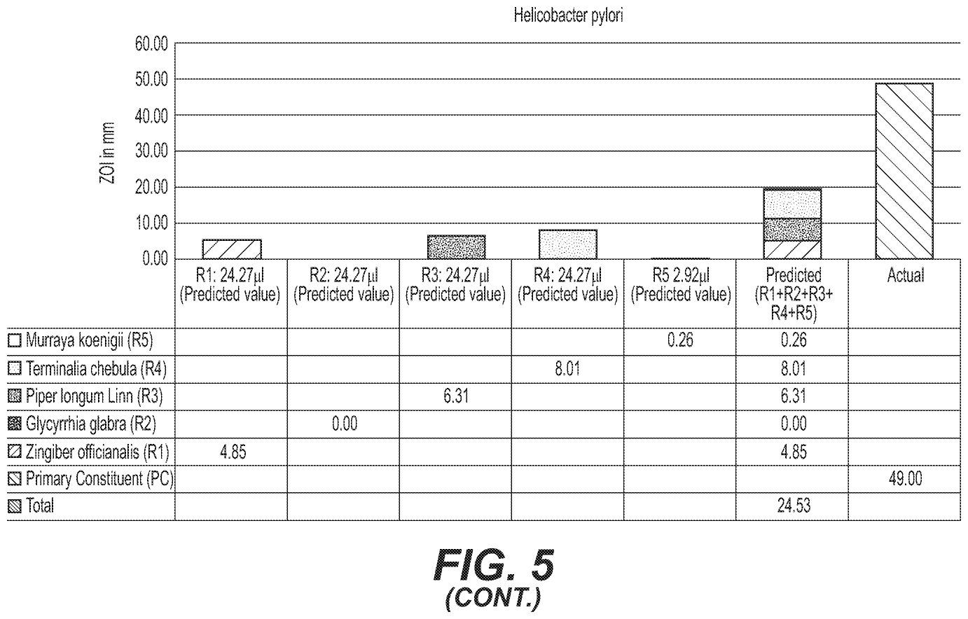

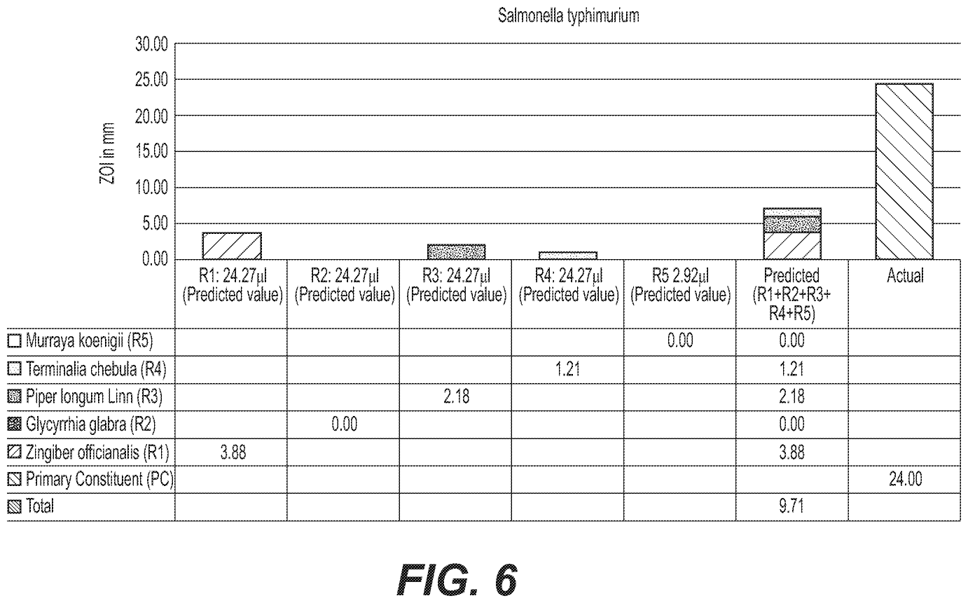

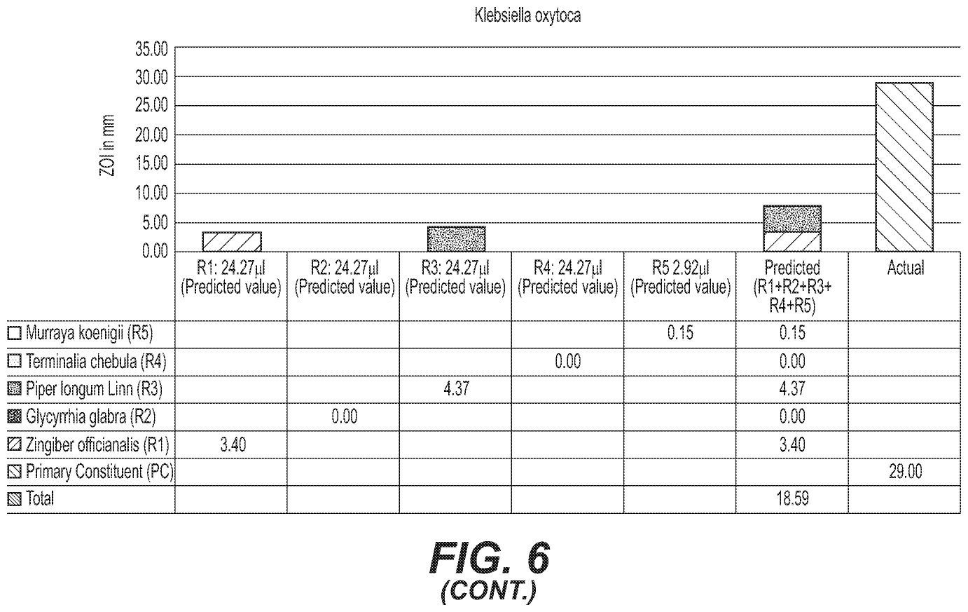

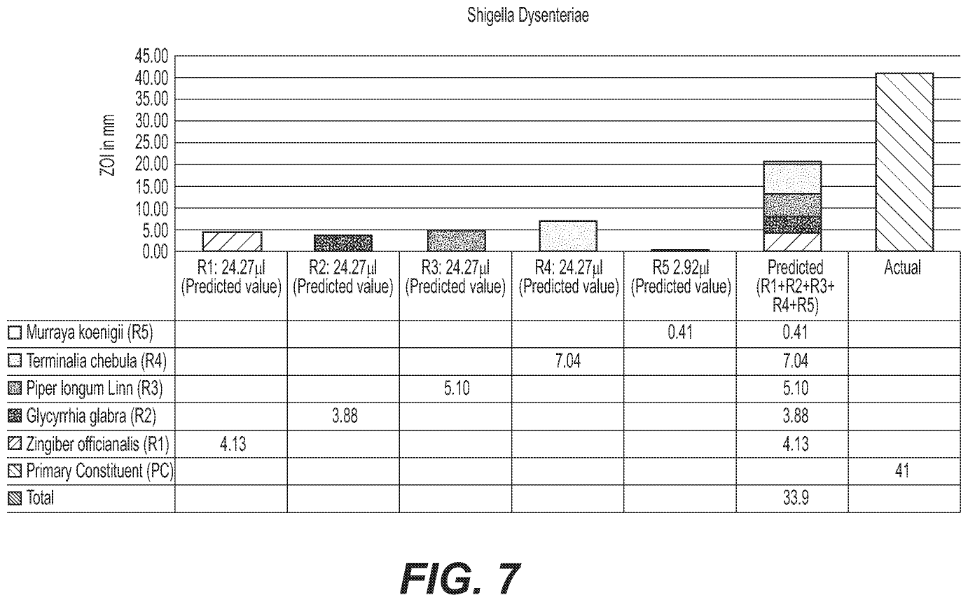

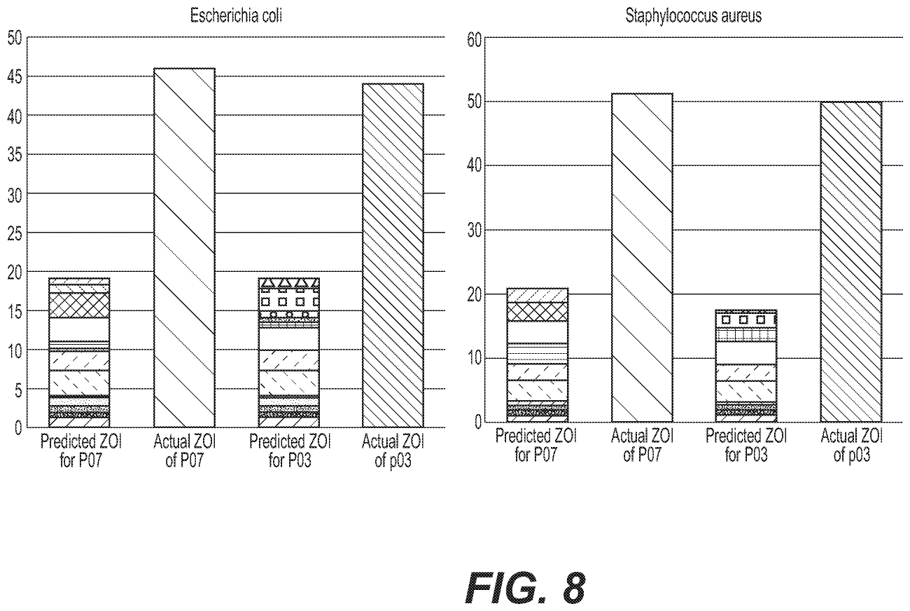

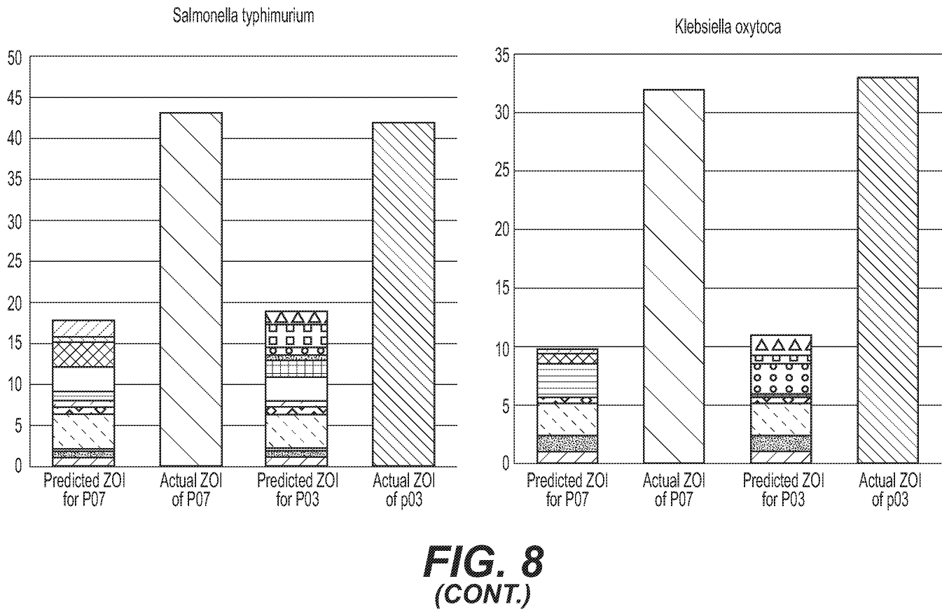

Similar to the above calculation, the graphs of FIG. 4 for Escherichia coli, FIG. 4 (CONT.) and FIG. 5, FIG. 5 (CONT.), FIG. 6, FIG. 6 (CONT.), FIG. 7, FIG. 8, FIG. (CONT.) and FIG. 9, FIG. (CONT.) provide predicted ZOI of individual extract calculated as above. The predicted ZOIs are added to obtain the total predicted activity of the final composition and a comparison with the actual ZOI of the primary constituents' composition is shown. It was observed that for each bacteria strain the actual value of ZOI was higher than the predicted value based on ZOI expected from stoichiometric amounts of the total of individual extracts in the composition. For the primary constituents' composition the improvement over the predicted value was more than 100% against each bacterial strain. FIG. 8 and FIG. 9 shows the comparison between predicted value of P07 and P03 against actual value. More than 100% improvement over the predicted value was observed for each bacterial strain. Whether the primary constituents' composition, the P07 or the P03, the entire compositions showed greater efficacy in ZOI than the expected based on stoichiometric amounts of individual plant extract.

### EXAMPLE 7

### In-Vitro Microbial Study.

The purpose of this illustration was to show the activity of each plant extract that make a medicinal composition individually against pathogenic microbes and beneficial microbes.

Anti-bacterial activity of each extract was assessed in-vitro against, Escherichia coli (P1), Staphylococcus aureus (P2), Salmonella typhimurium (P3), Klebsiella oxytoca (P4), Vibrio cholera (P5), Helicobacter pylori (P6), and Shigella dysenteriae (P7). Activity was also assessed against beneficial microbes such as; Lactobacillus acidophilus (BB1), Streptococcus thermophilus (BB2), Bacteroides fragilis (BB3), Clostridium butyricum (BB4), Faecalibacterium prausnitzii (BB5).

Faecali-bacterium prausnitzii by resazurin dye reduction method (Sarker S D, Nahar L, Kumarasamy Y. Microtitre plate-based antibacterial assay incorporating resazurin as an indicator of cell growth, and its application in the in-vitro antibacterial screening of phytochemicals, Methods. 2007 August; 42(4): 321-324).

The sample selected for study are Piper longum Linn, Terminalia, chebula Retz, Murraya koenigii, Zingiber officinale, Glycyrrhiza glabra, Curcuma longa, Centella asiatica, Cyperus rotundus, Tinospora cordifolia, Hemidesmus indicus, Andrographis paniculata, Alpinia galanga, Boerhavia diffusa, and Cassia fistula, along with them Primary constituents', P03 and P07 composition made as per example 5. Primary constituents' composition comprises of Piper longum Linn, Terminalia chebula, Murraya koenigii, Zingiber officinale and Glycyrrhiza glabra.

Each sample is tested separately against the selected bacteria strains to determine their Minimum Inhibitory Concentration for each bacteria strain. A sterile 96 well (8×12) plate was prepared and labeled under aseptic conditions. Stock solution of test sample was prepared by dissolving 100 mg powdered sample in DMSO to get concentration 10 mg/ml.

A volume of 100 μl of test sample solution was pipette into the first row of the plate. To all other wells 50 μl of nutrient broth was added.

Serial dilutions were performed using a multichannel pipette to get final concentrations from 1000 μg/ml to 7.81 μg/ml i.e. (1000, 500, 250, 125, 62.5, 31.25, 15.625 and 7. 8125 μg/ml).

To each well 10 μl of resazurin indicator solution and 30 μl of 3× strength isosensitized broth was added. Finally, 10 μl of microbial suspension (5×10⁶ cfu/mL) was added to each well to achieve a concentration of 5×10⁵ cfu/mL.

Each plate was wrapped loosely with cling film to ensure that cultures did not become dehydrated. Each Plate had a set of positive (with organism), negative (without organism) and a standard (ciprofloxacin).

The plates were prepared and placed in an incubator at 37° C. for 18-24 h. The colour change was then assessed visually. Any colour changes from purple to pink or colourless were recorded as positive for bacterial growth. The lowest concentration at which no colour change occur (absence of growth) was taken as the MIC (Minimum Inhibitory Concentration) value.

Importantly, the significant antibacterial activity was exhibited against unfavorable bacteria at a lower concentration, with a lower MIC values (15.625-250 μg/ml) in contrast activity against favorable bacteria required higher concentration, MIC values (250-1000 μg/ml).

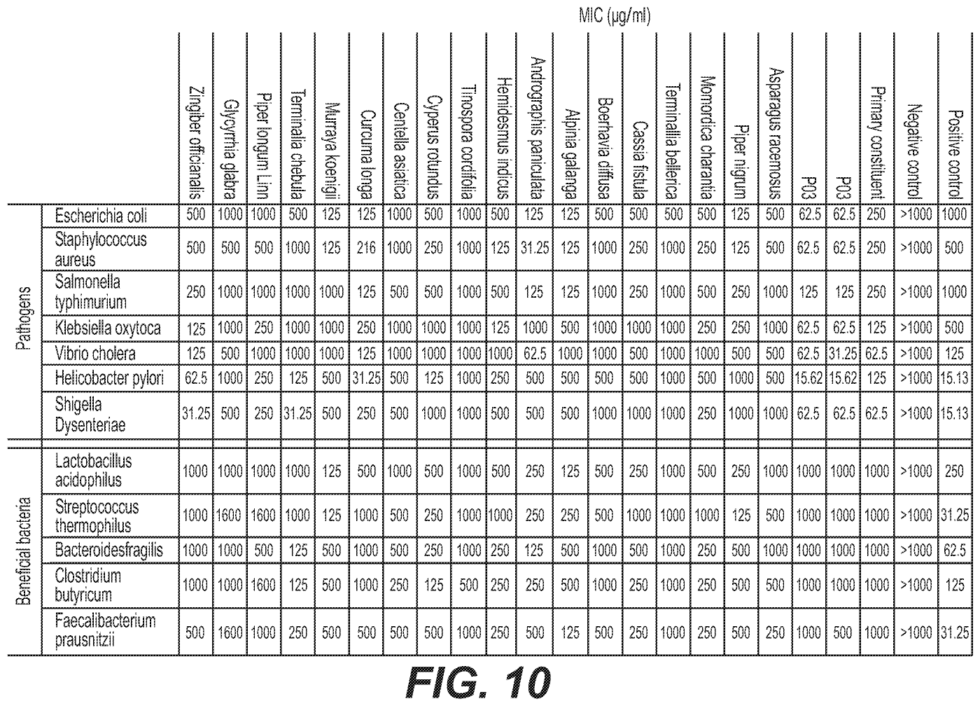

The MIC on each of the individual plant extract was provided in FIG. 10. From those data an expected value of MIC for primary constituents' composition, P03 and P07 was determined. A comparison of expected predicted value and actual experimental value is provided in FIG. 10 to FIG. 13.

The minimum concentration required for inhibiting the growth of pathogenic microbes was much less for Primary constituents' compoaition, P03 and P07 compared to their predicted value.

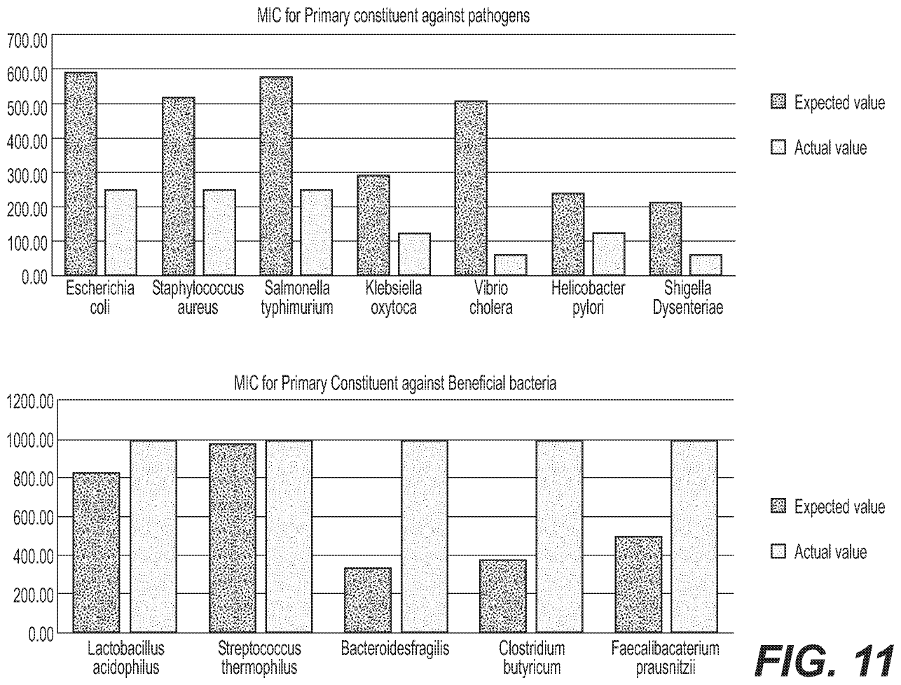

In FIG. 11 MIC requirement for primary constituents' composition against pathogenic bacteria and beneficial bacteria was provided. The minimum concentration for inhibiting any pathogenic bacteria was observed to be less than 300 μg/ml, which was clinically significant. On the other hand the primary constituents' composition did not inhibit beneficial bacteria at such low concentration at which it inhibits pathogens. Comparing the predicted (or expected) value and actual value for primary constituents' composition it was observed that the concentration required in actual was less than the predicted value, much less than predicted, less than half for few bacteria. This signifies a present of synergetic effect among the constituents of the composition.

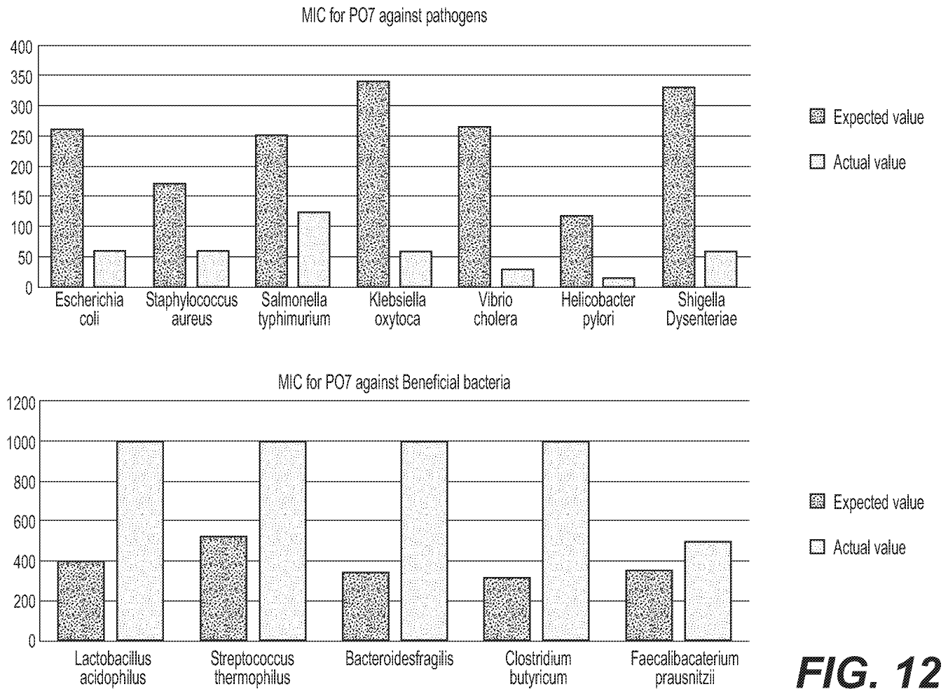

FIG. 12 shows the MIC of P07 against beneficial and pathogenic bacteria strain and the actual value against the predicted MIC value are compared. It was found out that the overall MIC for P07 against pathogenic bacteria was less than 150 μg/ml, which was clinically significant. Compared to the predicted/expected value the minimum concentration required in actual against pathogenic bacteria was significantly less, less than one fourth in most cases, except for Staphylococcus aureus and Salmonella typhimurium. This signifies a present of synergetic effect among the constituents of the composition. Even for Staphylococcus aureus and Salmonella typhimurium the actual MIC was less than half the predicted MIC value. P07 did not show any inhibition of beneficial bacteria at clinically significant concentration of less than 300 μg/ml.

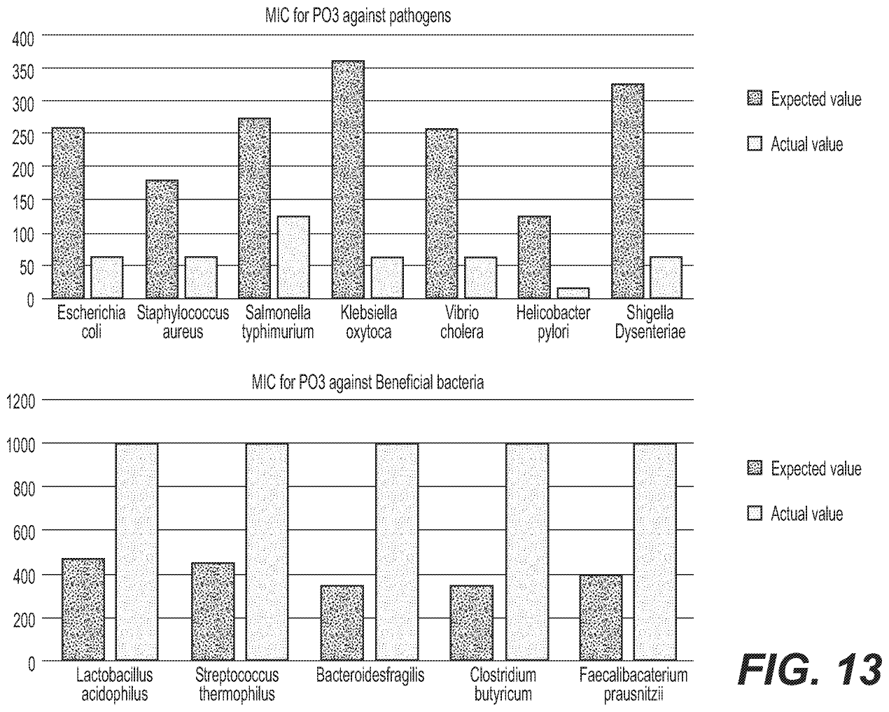

FIG. 13 shows the MIC requirement of P03 and comparing the expected value against the predicted MIC value. It was found out that the overall MIC for P03 against pathogenic bacteria was less than 150 μg/ml, which was clinically significant. Compared to the predicted/expected value the minimum concentration required in actual against pathogenic bacteria was significantly less, less than one fourth in most cases, except for Staphylococcus aureus and Salmonella typhimurium. This signifies a present of synergetic effect among the constituents of the composition. Even for Staphylococcus aureus and Salmonella typhimurium the actual MIC is less than half the predicted MIC value. P03 did not show any inhibition of beneficial bacteria at clinically significant concentration of less than 300 μg/ml. The predicted value was calculated through the individual plant extract activity provided in FIG. 9.

For Primary constituents' composition, P03 and P07 the MIC did not even pass 300 μg/ml, which shows they show clinically significant inhibition of pathogenic bacteria at lower concentration. At the same time, Primary constituents' composition, P03 and P07 did not show any adverse effects on the beneficial bacteria strains. A MIC value above 300 μg/ml was a good sign that there is no adverse effect, and for Primary constituents' composition, P03 and P07 the MIC was recorded to be above or equal to 500 μg/ml.

What is claimed is:

1. A gastrointestinal health enhancing composition comprising an extract of Piper longum, an extract of Terminalia chebula, an extract of Murraya koenigii, an extract Zingiber officinale and an extract Glycyrrhiza glabra.

2. The gastrointestinal health enhancing composition of claim 1 comprising by weight:
about 20% to about 30% of the extract of Piper longum,
about 20% to about 30% of the extract of Terminalia chebula,
about 20% to about 30% of extract of Zingiber officinale,
about 20% to about 30% of extract of Glycyrrhiza glabra, and,
about 1% to about 5% of extract of Murraya koenigii.

3. The gastrointestinal health enhancing composition of claim 1 comprising a weight ratio of 1:1:1:1 of the extract of Piper longum: the extract of Terminalia chebula: the extract of Zingiber officinale: the extract of Glycyrrhiza glabra.

4. The gastrointestinal health enhancing composition of claim 1 comprising a weight ratio of the extract of Piper longum to extract of Murraya koenigii ranging from about 5:1 to about 25:1.

5. The gastrointestinal health enhancing composition of claim 1 comprising a weight ratio of extract of Terminalia chebula to extract of Murraya koenigii ranging from about 5:1 to about 25:1.

6. The gastrointestinal health enhancing composition of claim 1 comprising a weight ratio of extract of Zingiber officinale to extract of Murraya koenigii ranging from about 5:1 to about 25:1.

7. The gastrointestinal health enhancing composition of claim 1 comprising a weight ratio of extract of Glycyrrhiza glabra to extract of Murraya koenigii ranging from about 5:1 to about 25:1.

8. The gastrointestinal health enhancing composition of claim 1 comprising about 1% to about 10% alkaloids.

9. The gastrointestinal health enhancing composition of claim 1 comprising about 1% to about 10% gallic acid.

10. The gastrointestinal health enhancing composition of claim 1 comprising about 10% to about 30% polyphenols.

11. The gastrointestinal health enhancing composition of claim 1 comprising about 0.1% to about 10% glycyrrhizic acid.

12. The gastrointestinal health enhancing composition of claim 1 comprising about0.1% to about 10% total gingerols.

13. The gastrointestinal health enhancing composition of claim 1 comprising about 15% polyphenols.

14. The gastrointestinal health enhancing composition of claim 1 further comprising probiotics.

15. The gastrointestinal health enhancing composition of claim 1 further comprising an extract selected from the group consisting of an extract of Curcuma longa, an extract of Centella asiatica, an extract of Cyperus rotundus, an extract of Alpinia galangal, an extract of Acorus calamus, an extract of Tinospora cordifolia, an extract of Hemidesmus indicus, an extract of Cinnamomum tamala, an extract of Terminallia bellerica, an extract of Momordica charantia, an extract of Piper nigrum, an extract of Asparagus racemosus, an extract of Andrographis paniculate, an extract of Pterocarpus marsupium, an extract of Swertia chirata Buch, an extract of Boerhavia diffusa, and combinations thereof.

16. The gastrointestinal health enhancing composition of claim 15 further comprising an extract selected from the group consisting of an extract of Ajowa, an extract of Coriander satuvum, an extract of Asparagus officinalis, an extract of Camellia sinensis, an extract of Ocimum sanctum, an extract of Sesamum indicum, an extract of Punica granatum, an extract of Emblica officinalis, an extract of Phyllanthus niruri, an extract of Cassia fistula, and combinations thereof.

17. The gastrointestinal health enhancing composition of claim 1 further comprising an extract selected from the group consisting of an extract of Curcuma longa, an extract of Centella asiatica, an extract of Cyperus rotundus, an extract of Terminallia bellerica, an extract of Hemidesmus indicus, an extract of Piper nigrum, an extract of Asparagus racemosus, an extract of Andrographis paniculata, an extract of Alpinia galangal, and combinations thereof.

18. The gastrointestinal health enhancing composition of claim 17 comprising by weight:
about 5% to about 15% of the extract of Piper longum,
about 1% to about 11% of the extract of Terminalia chebula,
about 1% to about 7% of the extract of Murraya koenigii,
about 5% to about 15% of the extract of Zingiber officianale,
about 1% to about 11% of the extract of Glycyrrhiza glabra,
about 5% to about 15% of an extract of Curcuma longa,
about 1% to about 11% of the extract of Centella asiatica,
about 1% to about 11% of the extract of Cyperus rotundus,
about 1% to about 11% of the extract of Terminallia bellerica,
about 1% to about 11% of the extract of Hemidesmus indicus,
about 1% to about 11% of the extract of Piper nigrum,
about 1% to about 11% of the extract of Asparagus racemosus,
about 1% to about 11% of the extract of Andrographis paniculata,
about 1% to about 11% of the extract of Alpinia galangal.

19. The gastrointestinal health enhancing composition of claim 1 further comprising an extract selected from the group consisting of an extract of Curcuma longa, an extract of Centella asiatica, an extract of Cyperus rotundus, an extract of Tinospora cordifolia, an extract of Hemidesmus indicus, an extract of Andrographis paniculata, an extract of Alpinia galanga, an extract of Boerhavia diffusa, an extract of Cassia fistula, and combinations thereof.

20. The gastrointestinal health enhancing composition of claim 19 comprising by weight:
about 1% to about 11% of the extract of Piper longum,
about 1% to about 11% of the extract of Terminalia chebula,
about 1% to about 11% of the extract of Murraya koenigii,
about 1% to about 11% of the extract of Zingiber officinale,
about 1% to about 11% of the extract of Glycyrrhiza glabra,
about 1% to about 11% of the extract of Curcuma longa,
about 1% to about 11% of the extract of Centella asiatica,
about 1% to about 11% of the extract of Cyperus rotundus,
about 1% to about 11% of the extract of Tinospora cordifolia,
about 1% to about 11% of the extract of Hemidesmus indicus,
about 1% to about 11% of the extract of Andrographis paniculata,
about 1% to about 11% of the extract of Alpinia galanga,
about 1% to about 11% of the extract of Boerhavia diffusa, and
about 1% to about 11% of the extract of Cassia fistula.

21. The gastrointestinal health enhancing composition of claim 1, wherein the extract of Piper longum is prepared from fruit of Piper longum.

22. The gastrointestinal health enhancing composition of claim 1, wherein the extract of Terminalia chebula is prepared from fruit of Terminalia chebula.

23. The gastrointestinal health enhancing composition of claim 1, wherein the extract of Murraya koenigii is prepared from leaf of Murraya koenigii.

24. The gastrointestinal health enhancing composition of claim 1, wherein the extract of Zingiber officinale is prepared from rhizomes of Zingiber officinale.

25. The gastrointestinal health enhancing composition of claim 1, wherein the extract of Glycyrrhiza glabra is prepared from rhizomes of Glycyrrhiza glabra.

26. A dosage form comprising the gastrointestinal health enhancing composition of claim 1, the dosage form selected from the group consisting of capsule, tablet, mini tablet, granule, sachet, powder, paste, ampoule, solution, suspension, emulsion, pills or cream.

27. A product comprising the gastrointestinal health enhancing composition of claim 1, the product selected from the group consisting of cookies, bread, and health supplement drinks.

28. A method of treating a gastrointestinal disorder comprising administering the gastrointestinal health enhancing composition of claim 1 to a subject in need thereof, wherein the gastrointestinal disorder is selected from the group consisting of abdominal pain, gastric bloating (abdominal distension), nausea/vomiting, eructation, flatus, borborygmus, acid reflux, tightness in upper abdomen, heart burn, pain while passing stools or incomplete evacuation, inability to control the urgency of passing stools, difficulty in passage of stools, constipation, diarrhea syndrome, and indigestion syndrome.

29. A method of improving quality of life comprising administering the gastrointestinal health enhancing composition of claim 1 to a subject in need thereof, wherein an improvement in the quality of life of the subject is assessed by improvement in a condition selected from the group consisting of abdominal pain, gastric bloating, abdominal distension, nausea/vomiting, eructation, flatus, borborygmus, acid reflux, tightness in upper abdomen, heart Burn, pain while passing stools or incomplete evacuation, inability to control the urgency of passing stools, difficulty in passage of stools, constipation, diarrhea syndrome, and indigestion syndrome.

30. A method of improving quality of life by administering the gastrointestinal health enhancing composition of claim 1 to a subject in need thereof, wherein an improvement in the quality of life of the subject is assessed by questionnaire based on a scale selected from the group consisting of gastrointestinal symptom rating scale, world health organization quality of life and patient assessment of constipation-quality of life.

31. A method of preparing a first gastrointestinal health enhancing composition, the method comprising:
a) collecting fresh rhizomes of Zingiber officinale and roots of Glycyrrhiza glabra;
washing the rhizomes and chopping into flakes;
drying the flakes to obtain dried flakes;
extracting dried flakes with 90% methanol and collecting the solvent part of methanol extraction;
concentrating the solvent part of the methanol extraction and drying to obtain a powder of an alcoholic extract of Zingiber officinale, and Glycyrrhiza glabra;
b) collecting fresh fruits of Piper longum and Terminalia chebula;
washing and chopping the fresh fruits into flakes;
drying the flakes;
extracting the flakes with 95% methanol and collecting the solvent part of the methanol extraction;
filtering and drying the solvent part of the methanol extraction to obtain a powder of an alcoholic extract of fruits of Piper longum and Terminalia chebula;
c) collecting fresh leaves of Murraya koenigii;
washing and chopping the fresh leaves to obtain chopped leaves;
drying the chopped leaves to obtain dried flakes;
extracting the dried flakes with 95% methanol and collecting the solvent part of the methanol extraction;
filtering, concentrating and drying the solvent part of the methanol extraction to obtain a powder of an alcoholic extract of leaves of Murraya koenigii;
d) blending the powder of the alcoholic extract of Zingiber officinale; and Glycyrrhiza glabra of step a), the powder of powder of the alcoholic extract of fruits of Piper longum and Terminalia chebula of step b) and the powder of the alcoholic extract of leaves of Murraya koenigii to obtain the first gastrointestinal health enhancing composition.

32. The first gastrointestinal health enhancing composition prepared by the method of claim 31.

33. The method of preparing the first gastrointestinal health enhancing composition of claim 31, further comprising a second method of preparing a second gastrointestinal health enhancing composition, the second method comprising:
collecting fresh rhizomes of Centella asiatica, Curcuma longa, Cyperus rotundus, and Alpinia galangal,
washing the rhizomes and chopping into flakes;
drying the flakes to obtain dried flakes;
extracting dried flakes with 90% methanol and collecting the solvent part of methanol extraction;
concentrating the solvent part of the methanol extraction and drying to obtain a powder of an alcoholic extract of Centella asiatica, Curcuma longa, Cyperus rotundus, and Alpinia galangal;
collecting fresh stem, bark and leaf of Tinospora cordifolia, Andrographis paniculata, Cassia fistula, Boerhaavia diffusa, and Centella asiatica;
washing the fresh stem, bark and leaf and chopping into flakes;
drying the flakes to obtain dried flakes;
extracting dried flakes with 90% methanol and collecting the solvent part of methanol extraction;
concentrating the solvent part of the methanol extraction and drying to obtain a powder of an alcoholic extract of fresh stems, bark and leaves of Tinospora cordifolia, Andrographis paniculata, Cassia fistula, Boerhaavia diffusa, and Centella asiatica,
mixing the powder of the alcoholic extract of Centella asiatica, Curcuma longa, Cyperus rotundus, and Alpinia galangal with the powder of the alcoholic extract of fresh stems, bark and leaves of Tinospora cordifolia, Andrographis paniculata, Cassia fistula, Boerhaavia diffusa, and Centella asiatica to obtain a mixture; and,
blending the first gastrointestinal health enhancing composition of claim 31 with the mixture to obtain the second gastrointestinal health enhancing composition.

34. The second gastrointestinal health enhancing composition prepared by the method of claim 33.

35. A method of preparing a gastrointestinal health enhancing composition, the method comprising:
a) collecting a plant part selected from the group consisting of leaf, stem, bark, rhizome, root, fruit and combinations thereof;
washing the plant part and chopping into flakes;
drying the flakes to obtain dried flakes;
extracting dried flakes with a solvent and collecting the solvent part after extraction;
concentrating the solvent part and drying to obtain a powder of an extract of the plant part,
wherein the leaf is selected from the group consisting of Murraya Koenigii, Boerhaavia diffusa, Centella asiatica, Andrographis paniculata,
wherein the stem is selected from the group consisting of Tinospora cordifolia, Andrographis paniculata,
wherein the bark is Cassia fistula,
wherein the rhizome is selected from the group consisting of Hemidesmus indicus, Cyperus rotundus, Alpinia galanga, Curcuma longa, and Zingiber officinale,
wherein the root is Glycyrrhiza glabra,
wherein the fruit is selected from the group consisting of Piper longum and Terminalia chebula,
wherein the solvent selected from the group consisting of hydroalcoholic 70% methanol and ethyl acetate,
wherein the powder of the extract is selected from the group consisting of an extract of the leaf of Murraya koenigii, an extract of the leaf of Boerhaavia diffusa, an extract of the leaf of Centella asiatica, an extract of the leaf of Andrographis paniculata, an extract of the stem of Tinospora cordifolia, an extract of the stem of Andrographis paniculata, an extract of the bark of Cassia fistula, an extract of the rhizome of Hemidesmus indicus, an extract of the rhizome of Cyperus rotundus, an extract of the rhizome of Alpinia galanga, an extract of the rhizome of Curcuma longa, an extract of the rhizome of Zingiber officinale, an extract of the root of Glycyrrhiza glabra, an extract of fruit of Piper longum, and an extract of fruit of Terminalia chebula,
wherein a first extract is obtained by combining the extract of leaf of Murraya koenigii, extract of the rhizome of Zingiber officinale, and an extract of the root of Glycyrrhiza glabra, an extract of fruit of Piper longum, and an extract of fruit of Terminalia chebula.

36. The first extract prepared by the method of claim 35.

37. The method of claim 33, wherein a second extract is prepared by combining an extract of the leaf of Murraya koenigii, an extract of the leaf of Boerhaavia diffusa, an extract of the leaf of Centella asiatica, an extract of the leaf of Andrographis paniculata, an extract of the stem of Tinospora cordifolia, an extract of the stem of Andrographis paniculata, an extract of the bark of Cassia fistula, an extract of the rhizome of Hemidesmus indicus, an extract of the rhizome of Cyperus rotundus, an extract of the rhizome of Alpinia galanga, an extract of the rhizome of Curcuma longa, an extract of the rhizome of Zingiber officinale, an extract of the root of Glycyrrhiza glabra, an extract of fruit of Piper longum, and an extract of fruit of Terminalia chebula,

38. The second extract prepared by the method of claim 37.

39. The method of claim 34, wherein a third extract is prepared by combining the extract of the leaf of Murraya koenigii, an extract of the leaf of Centella asiatica, an extract of the leaf of Andrographis paniculata, an extract of the stem of Andrographis paniculata, an extract of the bark of Cassia fistula, an extract of the rhizome of Cyperus rotundus, an extract of the rhizome of Curcuma longa, an extract of the rhizome of Zingiber officinale, an extract of the root of Glycyrrhiza glabra, an extract of fruit of Piper longum, an extract of fruit of Terminalia chebula, an extract of fruit of Terminalia bellerica, an extract of fruit of Momordica charantica, an extract of fruit of Piper nigrum, and an extract of root of Asparagus racemosus.

40. The third extract prepared by the method of claim 39.
